(19) United States
(12) Patent Application Publication
(10) Pub. No.: US 2002/0019009 A1
(43) Pub. Date: Feb. 14, 2002

(54) High throughput screening (HTS) assays

(76) Inventors: Erwin Ludo Roggen, Lyngby (DK); Steffen Ernst, Broenshoej (DK); Henrik Pedersen, Bagsvaerd (DK)

Correspondence Address: NOVOZYMES NORTH AMERICA, INC., C/O NOVO NORDISK OF NORTH AMERICA, INC., 405 LEXINGTON AVENUE, SUITE 6400, NEW YORK, NY, 10174, US

(21) Appl. No.: 09/733,485

(22) Filed: Dec. 8, 2000

Related U.S. Application Data
(60) Provisional application No. 60/170,743, filed on Dec. 14, 1999.

(30) Foreign Application Priority Data
Dec. 9, 1999 (DK) PA 1999 01765

(51) Int. Cl. G01N 33/53; G01N 33/567; C12Q 1/68

(52) U.S. Cl. 435/7.1; 435/6; 435/7.21

(57) ABSTRACT

The present invention is to provide a method to perform assays that efficiently and accurately can screen large numbers of cell populations producing variants of a molecule of interest.

The present invention relates more specifically to a method for screening a library of protein variants for functional variants with reduced antibody binding capacity, comprising the steps of:

(i) generating a diversified library of protein variants starting from a relevant protein backbone,

(ii) transforming the library into suitable host cells,

(iii) culturing host cells,

(iv) sampling each cell culture,

(v) analyzing a sample by determining the antibody binding capacity of the variant protein,

(vi) analyzing a sample by determining the functionality of the variant protein.

### FIELD OF INVENTION

This invention relates to methods for screening libraries of protein variants for those having reduced immunogenicity as compared to a protein backbone. More specifically, the present invention provides a method for identifying protein variants with reduced immunogenicity in an efficient manner.

### BACKGROUND OF THE INVENTION

An increasing number of proteins, including enzymes, are being produced industrially, for use in various industries, housekeeping and medicine. Being proteins they are likely to stimulate an immunological response in man and animals, including an allergic response.

In the present context the terms allergic response, allergy, allergenic and allergenicity are used according to their usual definitions, i.e. to describe the reaction due to immune responses wherein the antibody most often is IgE, less often IgG4 and diseases due to this immune response. Allergic diseases include urticaria, hay-fever, asthma, and atopic dermatitis. They may even evolve into an anaphylactic shock.

Prevention of allergy in susceptible individuals is therefore a research area of great importance. Depending on the application, individuals get sensitized to the respective allergens by inhalation, direct contact with skin and eyes, or ingestion. The general mechanism behind an allergic response is divided in a sensitization phase and a symptomatic phase. The sensitization phase involves a first exposure of an individual to an allergen. This event activates specific T- and B-lymphocytes, and leads to the production of allergen-specific IgE antibodies (in the present context the antibodies are denoted as usual, i.e. immunoglobulin E is IgE etc.). These IgE antibodies eventually facilitate allergen capturing and presentation to T-lymphocytes at the onset of the symptomatic phase. This phase is initiated by a second exposure to the same or a resembling antigen. The specific IgE antibodies bind to the specific IgE receptors on mast cells and basophils, among others, in a mode that allows antigen binding to the cell-bound IgE antibodies. The polyclonal nature of this process results in bridging and clustering of the IgE receptors, and subsequently in the activation of mast cells and basophiles. This activation triggers the release of various chemical mediators involved in the early as well as late phase reactions of the symptomatic phase of allergy.

Various attempts to reduce the immunogenicity of polypeptides and proteins have been conducted. It has been found that small changes in an epitope may affect the binding to an antibody. This may result in a reduced importance of such an epitope, maybe converting it from a high affinity to a low affinity epitope, or maybe even resulting in epitope loss, i.e. that the epitope cannot sufficiently bind an antibody to elicit an immunogenic response.

Technologies such as DNA shuffling, random DNA mutagenesis and in vivo recombination have allowed the generation of enormous populations of variant cells that produce variants of a certain protein. In addition, it has become possible in recombinant host strains to establish large libraries of natural enzymes cloned from other organisms. Together these technologies have created a need for assays that efficiently and accurately can screen large numbers of variants, high throughput screening (HTS) assays.

Most HTS methods are designed to detect an improved functionality of the expressed protein variants. In this case, however, we are interested in isolating variants with reduced antibody binding capacity, and hence we risk to select variants with debilitated functionality (which are also likely to bind antibodies poorly) or variants which expresses and/or secretes poorly and hence show very low antibody binding simply because they are not present in the supernatant. To overcome this limitation it is necessary to deviate from the design of most present HTS methods and introduce a dual assay system in which both antibody binding capacity and functionality are determined without compromising the high throughput capability necessary to benefit the diversity of diversified libraries.

The prior art, different publications have disclosed aspects that are useful for creating protein variants with altered antibody binding capacity, but none have disclosed a method for screening a diversified library expressed in host cells to search for functional protein variants with reduced antibody binding capacity.

For instance, WO 99/447680 discloses the modification of B-cell epitopes by protein engineering. However, the method is based on crystal structures of Fab-antigen complexes, and B-cell epitopes are defined as “a section of the surface of the antigen comprising 15-25 amino acid residues, which are within a distance from the atoms of the antibody enabling direct interaction” (p.3). This publication does not show how one selects which Fab fragment to use (e.g. to target the most dominant allergy epitopes) or how one selects the substitutions to be made. Further, their method cannot be used in the absence of such crystallographic data, which is very cumbersome, sometimes impossible, to obtain—especially since one would need a separate crystal structure for each epitope to be changed.

Slootstra et al; Molecular Diversity, 2, pp. 156-164, 1996 disclose the screening of a semirandom library of peptides for their binding properties to three monoclonal antibodies by immobilizing the peptides on polyethylene pins and binding a dilution series of each antibody to the pins. In this reference, all peptides are prepared by chemical synthesis; hence, it does not disclose any method to overcome background problems from using gene-encoded polypeptides expressed in a microbial host. Further, the antibody binding assays are based on 10-step dilution series for each antibody, meaning that 30 separate assays are necessary to evaluate each test compound. This makes the disclosed methods insufficient for high-throughput screening.

WO 97/30150 discloses the construction and expression of diversified libraries of a myelin basic protein (MBP) and the analysis of these variants by testing their T-cell antagonizing activity. This reference, however, only tests for ‘trans-dominant effects’ (p. 17) in which a single peptide harboring a productive mutation will show up even in the presence of 10 unchanged peptide fragments. This means that dysfunctional or poorly expressed protein variants will show no response and hence, that the teachings of this reference cannot be used for devising an assay to identify protein variants with reduced antibody binding capacity and retained functionality.

Below we describe a method of performing an assay that has been specifically developed for the screening of large populations of clones producing variants of a given protein.

The methods described below allow screening library of protein variants for functional variants with reduced antibody binding capacity.

### SUMMARY OF THE INVENTION

The problem to be solved by the present invention is to provide a method to perform assays that efficiently and accurately can screen large numbers of cell populations producing variants of a molecule of interest.

In a first aspect the invention relates to a method for high throughput screening (HTS) of a large population of host cells for production of a molecule of interest.

Specifically, the invention relates to a method for screening a library of protein variants for functional variants with reduced antibody binding capacity, comprising the steps of:

(i) generating a diversified library of protein variants starting from a relevant protein backbone,

(ii) transforming the library into suitable host cells,

(iii) culturing host cells,

(iv) sampling each cell culture,

(v) analysing a sample by determining the antibody binding capacity of the variant protein,

(vi) analysing a sample by determining the functionality of the variant protein.

Definitions

Prior to a discussion of the detailed embodiments of the invention, a definition of specific terms related to the main aspects of the invention is provided.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”) DNA Cloning: A Practical Approach, Volumes I and II /D. N. Glover ed. 1985); Oligonucleotide Synthesis (M. J. Gait ed. 1984); Nucleic Acid Hybridization (B. D. Hames & S. J. Higgins eds (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984).

When applied to a protein, the term “isolated” indicates that the protein is found in a condition other than its native environment, such as apart from blood and animal tissue. In a preferred form, the isolated protein is substantially free of other proteins, particularly other proteins of animal origin. It is preferred to provide the proteins in a highly purified form, i.e., greater than 95% pure, more preferably greater than 99% pure. When applied to a polynucleotide molecule, the term “isolated” indicates that the molecule is removed from its natural genetic milieu, and is thus free of other extraneous or unwanted coding sequences, and is in a form suitable for use within genetically engineered protein production systems. Such isolated molecules are those that are separated from their natural environment and include cDNA and genomic clones. Isolated DNA molecules of the present invention are free of other genes with which they are ordinarily associated, and may include naturally occurring 5′ and 3′ untranslated regions such as promoters and terminators. The identification of associated regions will be evident to one of ordinary skill in the art (see for example, Dynan and Tijan, Nature 316:774-78, 1985).

A “polynucleotide” is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”) in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary or quaternary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

A DNA “coding sequence” is a double-stranded DNA sequence, which is transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. If the coding sequence is intended for expression in a eukaryotic cell, a polyadenylation signal and transcription termination sequence will usually be located 3′ to the coding sequence.

An “Expression vector” is a DNA molecule, linear or circular, that comprises a segment encoding a polypeptide of interest operably linked to additional segments that provide for its transcription. Such additional segments may include promoter and terminator sequences, and optionally one or more origins of replication, one or more selectable markers, an enhancer, a polyadenylation signal, and the like. Expression vectors are generally derived from plasmid or viral DNA, or may contain elements of both.

Transcriptional and translational control sequences are DNA regulatory sequences, such as promoters, enhancers, terminators, and the like, that provide for the expression of a coding sequence in a host cell. In eukaryotic cells, polyadenylation signals are control sequences.

A “secretory signal sequence” is a DNA sequence that encodes a polypeptide (a “secretory peptide” that, as a component of a larger polypeptide, directs the larger polypeptide through a secretory pathway of a cell in which it is synthesized. The larger polypeptide is commonly cleaved to remove the secretory peptide during transit through the secretory pathway.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

“Operably linked”, when referring to DNA segments, indicates that the segments are arranged so that they function in concert for their intended purposes, e.g. transcription initiates in the promoter and proceeds through the coding segment to the terminator.

A coding sequence is “under the control” of transcriptional and translational control sequences in a cell when RNA polymerase transcribes the coding sequence into mRNA, which is then trans-RNA spliced and translated into the protein encoded by the coding sequence.

“Isolated polypeptide” is a polypeptide which is essentially free of other non-[enzyme] polypeptides, e.g., at least about 20% pure, preferably at least about 40% pure, more preferably about 60% pure, even more preferably about 80% pure, most preferably about 90% pure, and even most preferably about 95% pure, as determined by SDS-PAGE.

“Heterologous” DNA refers to DNA not naturally located in the cell, or in a chromosomal site of the cell. Preferably, the heterologous DNA includes a gene foreign to the cell.

A cell has been “transfected” by exogenous or heterologous DNA when such DNA has been introduced inside the cell. A cell has been “transformed” by exogenous or heterologous DNA when the transfected DNA effects a phenotypic change. Preferably, the transforming DNA should be integrated (covalently linked) into chromosomal DNA making up the genome of the cell.

A “clone” is a population of cells derived from a single cell or common ancestor by mitosis.

“Homologous recombination” refers to the insertion of a forreign DNA sequence of a vector in a chromosome. Preferably, the vector targets a specific chromosomal site for homologous recombination. For specific homologous recombination, the vector will contain sufficiently long regions of homology to sequences of the chromosome to allow complementary binding and incorporation of the vector into the chromosome. Longer regions of homology, and greater degrees of sequence similarity, may increase the efficiency of homologous recombination.

Nucleic Acid Sequence

The techniques used to isolate or clone a nucleic acid sequence encoding a polypeptide are known in the art and include isolation from genomic DNA, preparation from cDNA, or a combination thereof. The cloning of the nucleic acid sequences of the present invention from such genomic DNA can be effected, e.g., by using the well known polymerase chain reaction (PCR) or antibody screening of expression libraries to detect cloned DNA fragments with shared structural features. See, e.g., Innis et al., 1990, A Guide to Methods and Application, Academic Press, New York. Other nucleic acid amplification procedures such as ligase chain reaction (LCR), ligated activated transcription (LAT) and nuceic acid sequence-based amplification (NASBA) may be used. The nucleic acid sequence may be cloned from a strain producing the polypeptide, or from another related organism and thus, for example, may be an allelic or species variant of the polypeptide encoding region of the nucleic acid sequence.

The term “isolated” nucleic acid sequence as used herein refers to a nucleic acid sequence which is essentially free of other nucleic acid sequences, e.g., at least about 20% pure, preferably at least about 40% pure, more preferably about 60% pure, even more preferably about 80% pure, most preferably about 90% pure, and even most preferably about 95% pure, as determined by agarose gel electorphoresis. For example, an isolated nucleic acid sequence can be obtained by standard cloning procedures used in genetic engineering to relocate the nucleic acid sequence from its natural location to a different site where it will be reproduced. The cloning procedures may involve excision and isolation of a desired nucleic acid fragment comprising the nucleic acid sequence encoding the polypeptide, insertion of the fragment into a vector molecule, and incorporation of the recombinant vector into a host cell where multiple copies or clones of the nucleic acid sequence will be replicated. The nucleic acid sequence may be of genomic, cDNA, RNA, semisynthetic, synthetic origin, or any combinations thereof.

Nucleic Acid Construct

As used herein the term “nucleic acid construct” is intended to indicate any nucleic acid molecule of cDNA, genomic DNA, synthetic DNA or RNA origin. The term “construct” is intended to indicate a nucleic acid segment which may be single- or double-stranded, and which may be based on a complete or partial naturally occurring nucleotide sequence encoding a polypeptide of interest. The construct may optionally contain other nucleic acid segments.

The DNA of interest may suitably be of genomic or cDNA origin, for instance obtained by preparing a genomic or cDNA library and screening for DNA sequences coding for all or part of the polypeptide by hybridization using synthetic oligonucleotide probes in accordance with standard techniques (cf. Sambrook et al., supra).

The nucleic acid construct may also be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. According to the phosphoamidite method, oligonucleotides are synthesized, e.g. in an automatic DNA synthesizer, purified, annealed, ligated and cloned in suitable vectors.

Furthermore, the nucleic acid construct may be of mixed synthetic and genomic, mixed synthetic and or mixed genomic and cDNA origin prepared by ligating fragments of synthetic, genomic or cDNA origin (as appropriate), the fragments corresponding to various parts of the entire nucleic acid construct, in accordance with standard techniques.

The nucleic acid construct may also be prepared by polymerase chain reaction using specific primers, for instance as described in U.S. Pat. No. 4,683,202 or Saiki et al., Science 239 (1988), 487-491.

The term nucleic acid construct may be synonymous with the term expression cassette when the nucleic acid construct contains all the control sequences required for expression of a coding sequence of the present invention. The term “coding sequence” as defined herein is a sequence which is transcribed into mRNA and translated into a polypeptide of the present invention when placed under the control of the above mentioned control sequences. The boundaries of the coding sequence are generally determined by a translation start codon ATG at the 5′-terminus and a translation stop codon at the 3′-terminus. A coding sequence can include, but is not limited to, DNA, cDNA, and recombinant nucleic acid sequences.

The term “control sequences” is defined herein to include all components which are necessary or advantageous for expression of the coding sequence of the nucleic acid sequence. Each control sequence may be native or foreign to the nucleic acid sequence encoding the polypeptide. Such control sequences include, but are not limited to, a leader, a polyadenylation sequence, a propeptide sequence, a promoter, a signal sequence, and a transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleic acid sequence encoding a polypeptide.

The control sequence may be an appropriate promoter sequence, a nucleic acid sequence which is recognized by a host cell for expression of the nucleic acid sequence. The promoter sequence contains transcription and translation control sequences which mediate the expression of the polypeptide. The promoter may be any nucleic acid sequence which shows transcriptional activity in the host cell of choice and may be obtained from genes encoding extracellular or intracellular polypeptides either homologous or heterologous to the host cell. The control sequence may also be a suitable transcription terminator sequence, a sequence recognized by a host cell to terminate transcription. The terminator sequence is operably linked to the 3′ terminus of the nucleic acid sequence encoding the polypeptide. Any terminator which is functional in the host cell of choice may be used in the present invention.

The control sequence may also be a polyadenylation sequence, a sequence which is operably linked to the 3′ terminus of the nucleic acid sequence and which, when transcribed, is recognized by the host cell as a signal to add polyadenosine residues to transcribed mRNA. Any polyadenylation sequence which is functional in the host cell of choice may be used in the present invention.

The control sequence may also be a signal peptide coding region, which codes for an amino acid sequence linked to the amino terminus of the polypeptide which can direct the expressed polypeptide into the cell's secretory pathway of the host cell. The 5′ end of the coding sequence of the nucleic acid sequence may inherently contain a signal peptide coding region naturally linked in translation reading frame with the segment of the coding region which encodes the secreted polypeptide. Alternatively, the 5′ end of the coding sequence may contain a signal peptide coding region which is foreign to that portion of the coding sequence which encodes the secreted polypeptide. A foreign signal peptide coding region may be required where the coding sequence does not normally contain a signal peptide coding region. Alternatively, the foreign signal peptide coding region may simply replace the natural signal peptide coding region in order to obtain enhanced secretion relative to the natural signal peptide coding region normally associated with the coding sequence. The signal peptide coding region may be obtained from a glucoamylase or an amylase gene from an Aspergillus species, a lipase or proteinase gene from a Rhizomucor species, the gene for the alpha-factor from Saccharomyces cerevisiae, an amylase or a protease gene from a Bacillus species, or the calf preprochymosin gene. However, any signal peptide coding region capable of directing the expressed polypeptide into the secretory pathway of a host cell of choice may be used in the present invention.

The control sequence may also be a propeptide coding region, which codes for an amino acid sequence positioned at the amino terminus of a polypeptide. The resultant polypeptide is known as a proenzyme or propolypeptide (or a zymogen in some cases). A propolypeptide is generally inactive and can be converted to mature active polypeptide by catalytic or autocatalytic cleavage of the propeptide from the propolypeptide. The propeptide coding region may be obtained from the Bacillus subtilis alkaline protease gene (aprE), the Bacillus subtilis neutral protease gene (nprT), the Saccharomyces cerevisiae alpha-factor gene, or the Myceliophthora thermophilum laccase gene (WO 95/33836).

The nucleic acid constructs of the present invention may also comprise one or more nucleic acid sequences which encode one or more factors that are advantageous in the expression of the polypeptide, e.g., an activator (e.g., a trans-acting factor), a chaperone, and a processing protease. Any factor that is functional in the host cell of choice may be used in the present invention. The nucleic acids encoding one or more of these factors are not necessarily in tandem with the nucleic acid sequence encoding the polypeptide.

An activator is a protein which activates transcription of a nucleic acid sequence encoding a polypeptide (Kudla et al., 1990, EMBO Journal 9:1355-1364; Jarai and Buxton, 1994, Current Genetics 26:2238-244; Verdier, 1990, Yeast 6:271-297). The nucleic acid sequence encoding an activator may be obtained from the genes encoding Bacillus stearothermophilus NprA (nprA), Saccharomyces cerevisiae heme activator protein 1 (hap1), Saccharomyces cerevisiae galactose metabolizing protein 4 (gal4), and Aspergillus nidulans ammonia regulation protein (areA). For further examples, see Verdier, 1990, supra and MacKenzie et al., 1993, Journal of General Microbiology 139:2295-2307.

A chaperone is a protein which assists another polypeptide in folding properly (Hartl et al., 1994, TIBS 19:20-25; Bergeron et al., 1994, TIBS 19:124-128; Demolder et al., 1994, Journal of Biotechnology 32:179-189; Craig, 1993, Science 260:1902-1903; Gething and Sambrook, 1992, Nature 355:33-45; Puig and Gilbert, 1994, Journal of Biological Chemistry 269:7764-7771; Wang and Tsou, 1993, The FASEB Journal 7:1515-11157; Robinson et al., 1994, Bio/Technology 1:381-384). The nucleic acid sequence encoding a chaperone may be obtained from the genes encoding Bacillus subtilis GroE proteins, Aspergillus oryzae protein disulphide isomerase, Saccharomyces cerevisiae calnexin, Saccharomyces cerevisiae BiP/GRP78, and Saccharomyces cerevisiae Hsp70. For further examples, see Gething and Sambrook, 1992, supra, and Hartl et al., 1994, supra.

A processing protease is a protease that cleaves a propeptide to generate a mature biochemically active polypeptide (Enderlin and Ogrydziak, 1994, Yeast 10:67-79; Fuller et al., 1989, Proceedings of the National Academy of Sciences USA 86:1434-1438; Julius et al., 1984, Cell 37:1075-1089; Julius et al., 1983, Cell 32:839-852). The nucleic acid sequence encoding a processing protease may be obtained from the genes encoding Aspergillus niger Kex2, Saccharomyces cerevisiae dipeptidylaminopeptidase, Saccharomyces cerevisiae Kex2, and Yarrowia lipolytica dibasic processing endoprotease (xpr6).

It may also be desirable to add regulatory sequences which allow the regulation of the expression of the polypeptide relative to the growth of the host cell. Examples of regulatory systems are those which cause the expression of the gene to be turned on or off in response to a chemical or physical stimulus, including the presence of a regulatory compound. Regulatory systems in prokaryotic systems would include the lac, tac, and trp operator systems. In yeast, the ADH2 system or GAL1 system may be used. In filamentous fungi, the TAKA alpha-amylase promoter, Aspergillus niger glucoamylase promoter, and the Aspergillus oryzae glucoamylase promoter may be used as regulatory sequences. Other examples of regulatory sequences are those which allow for gene amplification. In eukaryotic systems, these include the dihydrofolate reductase gene which is amplified in the presence of methotrexate, and the metallothionein genes which are amplified with heavy metals. In these cases, the nucleic acid sequence encoding the polypeptide would be placed in tandem with the regulatory sequence.

Promoters

Examples of suitable promoters for directing the transcription of the nucleic acid constructs of the present invention, especially in a bacterial host cell, are the promoters obtained from the E. coli lac operon, the Streptomyces coelicolor agarase gene (dagA), the Bacillus subtilis levansucrase gene (sacB), the Bacillus subtilis alkaline protease gene, the Bacillus licheniformis alpha-amylase gene (amyL), the Bacillus stearothermophilus maltogenic amylase gene (amyM), the Bacillus amyloliquefaciens alpha-amylase gene (amyQ), the Bacillus amyloliquefaciens BAN amylase gene, the Bacillus licheniformis penicillinase gene (penP), the Bacillus subtilis xylA and xylB genes, and the prokaryotic beta-lactamase gene (Villa-Kamaroff et al., 1978, Proceedings of the National Academy of Sciences USA 75:3727-3731), as well as the tac promoter (DeBoer et al., 1983, Proceedings of the National Academy of Sciences USA 80:21-25), or the Bacillus pumilus xylosidase gene, or by the phage Lambda PR or PL promoters or the E. coli lac, trp or tac promoters. Further promoters are described in “Useful proteins from recombinant bacteria” in Scientific American, 1980, 242:74-94; and in Sambrook et al., 1989, supra.

Examples of suitable promoters for directing the transcription of the nucleic acid constructs of the present invention in a filamentous fungal host cell are promoters obtained from the genes encoding Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, Fusarium oxysporum trypsin-like protease (as described in U.S. Pat. No. 4,288,627, which is incorporated herein by reference), and hybrids thereof. Particularly preferred promoters for use in filamentous fungal host cells are the TAKA amylase, NA2-tpi (a hybrid of the promoters from the genes encoding Aspergillus niger neutral (-amylase and Aspergillus oryzae triose phosphate isomerase), and glaA promoters. Further suitable promoters for use in filamentous fungus host cells are the ADH3 promoter (McKnight et al., The EMBO J. 4 (1985), 2093-2099) or the tpiA promoter.

Examples of suitable promoters for use in yeast host cells include promoters from yeast glycolytic genes (Hitzeman et al., J. Biol. Chem. 255 (1980), 12073-12080; Alber and Kawasaki, J. Mol. Appl. Gen. 1 (1982), 419-434) or alcohol dehydrogenase genes (Young et al., in Genetic Engineering of Microorganisms for Chemicals (Hollaender et al, eds.), Plenum Press, New York, 1982), or the TPI1 (U.S. Pat. No. 4,599,311) or ADH2-4c (Russell et al., Nature 304 (1983), 652-654) promoters.

Further useful promoters are obtained from the Saccharomyces cerevisiae enolase (ENO-1) gene, the Saccharomyces cerevisiae galactokinase gene (GAL1), the Saccharomyces cerevisiae alcohol dehydrogenase/glyceraldehyde-3-phosphate dehydrogenase genes (ADH2/GAP), and the Saccharomyces cerevisiae 3-phosphoglycerate kinase gene. Other useful promoters for yeast host cells are described by Romanos et al., 1992, Yeast 8:423-488. In a mammalian host cell, useful promoters include viral promoters such as those from Simian Virus 40 (SV40), Rous sarcoma virus (RSV), adenovirus, and bovine papilloma virus (BPV).

Examples of suitable promoters for directing the transcription of the DNA encoding the polypeptide of the invention in mammalian cells are the SV40 promoter (Subramani et al., Mol. Cell Biol. 1 (1981), 854-864), the MT-1 (metallothionein gene) promoter (Palmiter et al., Science 222 (1983), 809-814) or the adenovirus 2 major late promoter.

An example of a suitable promoter for use in insect cells is the polyhedrin promoter (U.S. Pat. No. 4,745,051; Vasuvedan et al., FEBS Lett. 311, (1992) 7-11), the P10 promoter (J. M. Vlak et al., J. Gen. Virology 69, 1988, pp. 765-776), the Autographa califomica polyhedrosis virus basic protein promoter (EP 397 485), the baculovirus immediate early gene 1 promoter (U.S. Pat. No. 5,155,037; U.S. Pat. No. 5,162,222), or the baculovirus 39K delayed-early gene promoter (U.S. Pat. No. 5,155,037; U.S. Pat. No. 5,162,222).

Terminators

Preferred terminators for filamentous fungal host cells are obtained from the genes encoding Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. for fungal hosts) the TPI1 (Alber and Kawasaki, op. cit.) or ADH3 (McKnight et al., op. cit.) terminators.

Preferred terminators for yeast host cells are obtained from the genes encoding Saccharomyces cerevisiae enolase, Saccharomyces cerevisiae cytochrome C (CYC1), or Saccharomyces cerevisiae glyceraldehyde-3-phosphate dehydrogenase. Other useful terminators for yeast host cells are described by Romanos et al., 1992, supra.

Polyadenylation Signals

Preferred polyadenylation sequences for filamentous fungal host cells are obtained from the genes encoding Aspergillus oryzae TAKA amylase, Aspergillus niger glucoamylase, Aspergillus nidulans anthranilate synthase, and Aspergillus niger alpha-glucosidase.

Useful polyadenylation sequences for yeast host cells are described by Guo and Sherman, 1995, Molecular Cellular Biology 15:5983-5990.

Polyadenylation sequences are well known in the art for mammalian host cells such as SV40 or the adenovirus 5 Elb region.

Signal Sequences

An effective signal peptide coding region for bacterial host cells is the signal peptide coding region obtained from the maltogenic amylase gene from Bacillus NCIB 11837, the Bacillus stearothermophilus alpha-amylase gene, the Bacillus licheniformis subtilisin gene, the Bacillus licheniformis beta-lactamase gene, the Bacillus stearothermophilus neutral proteases genes (nprT, nprS, nprM), and the Bacillus subtilis PrsA gene. Further signal peptides are described by Simonen and Palva, 1993, Microbiological Reviews 57:109-137.

An effective signal peptide coding region for filamentous fungal host cells is the signal peptide coding region obtained from Aspergillus oryzae TAKA amylase gene, Aspergillus niger neutral amylase gene, the Rhizomucor miehei aspartic proteinase gene, the Humicola lanuginosa cellulase or lipase gene, or the Rhizomucor miehei lipase or protease gene, Aspergillus sp. amylase or glucoamylase, a gene encoding a Rhizomucor miehei lipase or protease. The signal peptide is preferably derived from a gene encoding A. oryzae TAKA amylase, A. niger neutral (-amylase, A. niger acid-stable amylase, or A. niger glucoamylase.

Useful signal peptides for yeast host cells are obtained from the genes for Saccharomyces cerevisiae a-factor and Saccharomyces cerevisiae invertase. Other useful signal peptide coding regions are described by Romanos et al., 1992, supra.

For secretion from yeast cells, the secretory signal sequence may encode any signal peptide which ensures efficient direction of the expressed polypeptide into the secretory pathway of the cell. The signal peptide may be naturally occurring signal peptide, or a functional part thereof, or it may be a synthetic peptide. Suitable signal peptides have been found to be the a-factor signal peptide (cf. U.S. Pat. No. 4,870,008), the signal peptide of mouse salivary amylase (cf. O. Hagenbuchle et al., Nature 289, 1981, pp. 643-646), a modified carboxypeptidase signal peptide (cf L. A. Valls et al., Cell 48, 1987, pp. 887-897), the yeast BAR1 signal peptide (cf. WO 87/02670), or the yeast aspartic protease 3 (YAP3) signal peptide (cf. M. Egel-Mitani et al., Yeast 6, 1990, pp. 127-137).

For efficient secretion in yeast, a sequence encoding a leader peptide may also be inserted downstream of the signal sequence and uptream of the DNA sequence encoding the polypeptide. The function of the leader peptide is to allow the expressed polypeptide to be directed from the endoplasmic reticulum to the Golgi apparatus and further to a secretory vesicle for secretion into the culture medium (i.e. exportation of the polypeptide across the cell wall or at least through the cellular membrane into the periplasmic space of the yeast cell). The leader peptide may be the yeast a-factor leader (the use of which is described in e.g. U.S. Pat. No. 4,546,082, EP 16 201, EP 123 294, EP 123 544 and EP 163 529). Alternatively, the leader peptide may be a synthetic leader peptide, which is to say a leader peptide not found in nature. Synthetic leader peptides may, for instance, be constructed as described in WO 89/02463 or WO 92/11378.

For use in insect cells, the signal peptide may conveniently be derived from an insect gene (cf. WO 90/05783), such as the lepidopteran Manduca sexta adipokinetic hormone precursor signal peptide (cf. U.S. Pat. No. 5,023,328).

Expression Vectors

The present invention also relates to recombinant expression vectors comprising a nucleic acid sequence of the present invention, a promoter, and transcriptional and translational stop signals. The various nucleic acid and control sequences described above may be joined together to produce a recombinant expression vector which may include one or more convenient restriction sites to allow for insertion or substitution of the nucleic acid sequence encoding the polypeptide at such sites. Alternatively, the nucleic acid sequence of the present invention may be expressed by inserting the nucleic acid sequence or a nucleic acid construct comprising the sequence into an appropriate vector for expression. In creating the expression vector, the coding sequence is located in the vector so that the coding sequence is operably linked with the appropriate control sequences for expression, and possibly secretion.

The recombinant expression vector may be any vector (e.g., a plasmid or virus) which can be conveniently subjected to recombinant DNA procedures and can bring about the expression of the nucleic acid sequence. The choice of the vector will typically depend on the compatibility of the vector with the host cell into which the vector is to be introduced. The vectors may be linear or closed circular plasmids. The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. The vector system may be a single vector or plasmid or two or more vectors or plasmids which together contain the total DNA to be introduced into the genome of the host cell, or a transposon.

The vectors of the present invention preferably contain one or more selectable markers which permit easy selection of transformed cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers which confer antibiotic resistance such as ampicillin, kanamycin, chloramphenicol, tetracycline, neomycin, hygromycin or methotrexate resistance. A frequently used mammalian marker is the dihydrofolate reductase gene (DHFR). Suitable markers for yeast host cells are ADE2, HIS3, LEU2, LYS2, MET3, TRP1, and URA3. A selectable marker for use in a filamentous fungal host cell may be selected from the group including, but not limited to, amdS (acetamidase), argB (ornithine carbamoyltransferase), bar (phosphinothricin acetyltransferase), hygB (hygromycin phosphotransferase), niaD (nitrate reductase), pyrG (orotidine-5′-phosphate decarboxylase), sC (sulfate adenyltransferase), trpc (anthranilate synthase), and glufosinate resistance markers, as well as equivalents from other species. Preferred for use in an Aspergillus cell are the amdS and pyrG markers of Aspergillus nidulans or Aspergillus oryzae and the bar marker of Streptomyces hygroscopicus. Furthermore, selection may be accomplished by co-transformation, e.g., as described in WO 91/17243, where the selectable marker is on a separate vector.

The vectors of the present invention preferably contain an element(s) that permits stable integration of the vector into the host cell genome or autonomous replication of the vector in the cell independent of the genome of the cell.

The vectors of the present invention may be integrated into the host cell genome when introduced into a host cell. For integration, the vector may rely on the nucleic acid sequence encoding the polypeptide or any other element of the vector for stable integration of the vector into the genome by homologous or nonhomologous recombination. Alternatively, the vector may contain additional nucleic acid sequences for directing integration by homologous recombination into the genome of the host cell. The additional nucleic acid sequences enable the vector to be integrated into the host cell genome at a precise location(s) in the chromosome(s). To increase the likelihood of integration at a precise location, the integrational elements should preferably contain a sufficient number of nucleic acids, such as 100 to 1,500 base pairs, preferably 400 to 1,500 base pairs, and most preferably 800 to 1,500 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleic acid sequences. On the other hand, the vector may be integrated into the genome of the host cell by non-homologous recombination. These nucleic acid sequences may be any sequence that is homologous with a target sequence in the genome of the host cell, and, furthermore, may be non-encoding or encoding sequences.

For autonomous replication, the vector may further comprise an origin of replication enabling the vector to replicate autonomously in the host cell in question. Examples of bacterial origins of replication are the origins of replication of plasmids pBR322, pUC19, pACYC177, pACYC184, pUB110, pE194, pTA1060, and pAMB1. Examples of origin of replications for use in a yeast host cell are the 2 micron origin of replication, the combination of CEN6 and ARS4, and the combination of CEN3 and ARS1. The origin of replication may be one having a mutation which makes its functioning temperature-sensitive in the host cell (see, e.g., Ehrlich, 1978, Proceedings of the National Academy of Sciences USA 75:1433).

More than one copy of a nucleic acid sequence encoding a polypeptide of the present invention may be inserted into the host cell to amplify expression of the nucleic acid sequence. Stable amplification of the nucleic acid sequence can be obtained by integrating at least one additional copy of the sequence into the host cell genome using methods well known in the art and selecting for transformants.

The procedures used to ligate the elements described above to construct the recombinant expression vectors of the present invention are well known to one skilled in the art (see, e.g., Sambrook et al., 1989, supra).

Host Cells

The present invention also relates to recombinant host cells, comprising a nucleic acid sequence of the invention, which are advantageously used in the recombinant production of the polypeptides. The term “host cell” encompasses any progeny of a parent cell which is not identical to the parent cell due to mutations that occur during replication.

The cell is preferably transformed with a vector comprising a nucleic acid sequence of the invention followed by integration of the vector into the host chromosome. “Transformation” means introducing a vector comprising a nucleic acid sequence of the present invention into a host cell so that the vector is maintained as a chromosomal integrant or as a self-replicating extra-chromosomal vector. Integration is generally considered to be an advantage as the nucleic acid sequence is more likely to be stably maintained in the cell. Integration of the vector into the host chromosome may occur by homologous or non-homologous recombination as described above.

The choice of a host cell will to a large extent depend upon the gene encoding the polypeptide and its source. The host cell may be a unicellular microorganism, e.g., a prokaryote, or a nonunicellular microorganism, e.g., a eukaryote. Useful unicellular cells are bacterial cells such as gram positive bacteria including, but not limited to, a Bacillus cell, e.g., Bacillus alkalophilus, Bacillus amyloliquefaciens, Bacillus brevis, Bacillus circulans, Bacillus coagulans, Bacillus lautus, Bacillus lentus, Bacillus licheniformis, Bacillus megaterium, Bacillus stearothermophilus, Bacillus subtilis, and Bacillus thuringiensis; or a Streptomyces cell, e.g., Streptomyces lividans or Streptomyces murinus, or gram negative bacteria such as E. coli and Pseudomonas sp. In a preferred embodiment, the bacterial host cell is a Bacillus lentus, Bacillus licheniformis, Bacillus stearothermophilus or Bacillus subtilis cell. The transformation of a bacterial host cell may, for instance, be effected by protoplast transformation (see, e.g., Chang and Cohen, 1979, Molecular General Genetics 168:111-115), by using competent cells (see, e.g., Young and Spizizin, 1961, Journal of Bacteriology 81:823-829, or Dubnar and Davidoff-Abelson, 1971, Journal of Molecular Biology 56:209-221), by electroporation (see, e.g., Shigekawa and Dower, 1988, Biotechniques 6:742-751), or by conjugation (see, e.g., Koehler and Thorne, 1987, Journal of Bacteriology 169:5771-5278).

The host cell may be a eukaryote, such as a mammalian cell, an insect cell, a plant cell or a fungal cell.

Useful mammalian cells include Chinese hamster ovary (CHO) cells, HeLa cells, baby hamster kidney (BHK) cells, COS cells, or any number of other immortalized cell lines available, e.g., from the American Type Culture Collection.

Examples of suitable mammalian cell lines are the COS (ATCC CRL 1650 and 1651), BHK (ATCC CRL 1632, 10314 and 1573, ATCC CCL 10), CHL (ATCC CCL39) or CHO (ATCC CCL 61) cell lines. Methods of transfecting mammalian cells and expressing DNA sequences introduced in the cells are described in e.g. Kaufman and Sharp, J. Mol. Biol. 159 (1982), 601-621; Southern and Berg, J. Mol. Appl. Genet. 1 (1982), 327-341; Loyter et al., Proc. Natl. Acad. Sci. USA 79 (1982), 422-426; Wigler et al., Cell 14 (1978), 725; Corsaro and Pearson, Somatic Cell Genetics 7 (1981), 603, Ausubel et al., Current Protocols in Molecular Biology, John Wiley and Sons, Inc., N.Y., 1987, Hawley-Nelson et al., Focus 15 (1993), 73; Ciccarone et al., Focus 15 (1993), 80; Graham and van der Eb, Virology 52 (1973), 456; and Neumann et al., EMBO J. 1 (1982), 841-845.

In a preferred embodiment, the host cell is a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra). Representative groups of Ascomycota include, e.g., Neurospora, Eupenicillium (=Penicillium), Emericella (=Aspergillus), Eurotium (=Aspergillus), and the true yeasts listed above. Examples of Basidiomycota include mushrooms, rusts, and smuts. Representative groups of Chytridiomycota include, e.g., Allomyces, Blastocladiella, Coelomomyces, and aquatic fungi. Representative groups of Oomycota include, e.g., Saprolegniomycetous aquatic fungi (water molds) such as Achlya. Examples of mitosporic fungi include Aspergillus, Penicillium, Candida, and Alternaria. Representative groups of Zygomycota include, e.g., Rhizopus and Mucor.

In a preferred embodiment, the fungal host cell is a yeast cell. “Yeast” as used herein includes ascosporogenous yeast (Endomycetales), basidiosporogenous yeast, and yeast belonging to the Fungi Imperfecti (Blastomycetes). The ascosporogenous yeasts are divided into the families Spermophthoraceae and Saccharomycetaceae. The latter is comprised of four subfamilies, Schizosaccharomycoideae (e.g., genus Schizosaccharomyces), Nadsonioideae, Lipomycoideae, and Saccharomycoideae (e.g., genera Pichia, Kluyveromyces and Saccharomyces). The basidiosporogenous yeasts include the genera Leucosporidim, Rhodosporidium, Sporidiobolus, Filobasidium, and Filobasidiella. Yeast belonging to the Fungi Imperfecti are divided into two families, Sporobolomycetaceae (e.g., genera Sorobolomyces and Bullera) and Cryptococcaceae (e.g., genus Candida). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9, 1980. The biology of yeast and manipulation of yeast genetics are well known in the art (see, e.g., Biochemistry and Genetics of Yeast, Bacil, M., Horecker, B. J., and Stopani, A. O. M., editors, 2nd edition, 1987; The Yeasts, Rose, A. H., and Harrison, J. S., editors, 2nd edition, 1987; and The Molecular Biology of the Yeast Saccharomyces, Strathem et al., editors, 1981).

The yeast host cell may be selected from a cell of a species of Candida, Kluyveromyces, Saccharomyces, Schizosaccharomyces, Candida, Pichia, Hansehula, or Yarrowia. In a preferred embodiment, the yeast host cell is a Saccharomyces carlsbergensis, Saccharomyces cerevisiae, Saccharomyces diastaticus, Saccharomyces douglasii, Saccharomyces kluyveri, Saccharomyces norbensis or Saccharomyces oviformis cell. Other useful yeast host cells are a Kluyveromyces lactis Kluyveromyces fragilis Hansehula polymorpha, Pichia pastoris Yarrowia lipolytica, Schizosaccharomyces pombe, Ustilgo maylis, Candida maltose, Pichia guillermondii and Pichia methanolio cell (cf. Gleeson et al., J. Gen. Microbiol. 132, 1986, pp. 3459-3465; U.S. Pat. No. 4,882,279 and U.S. Pat. No. 4,879,231).

In a preferred embodiment, the fungal host cell is a filamentous fungal cell. “Filamentous fungi” include all filamentous forms of the subdivision Eumycota and Oomycota (as defined by Hawksworth et al., 1995, supra). The filamentous fungi are characterized by a vegetative mycelium composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative. In a more preferred embodiment, the filamentous fungal host cell is a cell of a species of, but not limited to, Acremonium, Aspergillus, Fusarium, Humicola, Mucor, Myceliophthora, Neurospora, Penicillium, Thielavia, Tolypocladium, and Trichoderma or a teleomorph or synonym thereof. In an even more preferred embodiment, the filamentous fungal host cell is an Aspergillus cell. In another even more preferred embodiment, the filamentous fungal host cell is an Acremonium cell. In another even more preferred embodiment, the filamentous fungal host cell is a Fusarium cell. In another even more preferred embodiment, the filamentous fungal host cell is a Humicola cell. In another even more preferred embodiment, the filamentous fungal host cell is a Mucor cell. In another even more preferred embodiment, the filamentous fungal host cell is a Myceliophthora cell. In another even more preferred embodiment, the filamentous fungal host cell is a Neurospora cell. In another even more preferred embodiment, the filamentous fungal host cell is a Penicillium cell. In another even more preferred embodiment, the filamentous fungal host cell is a Thielavia cell. In another even more preferred embodiment, the filamentous fungal host cell is a Tolypocladium cell. In another even more preferred embodiment, the filamentous fungal host cell is a Trichoderma cell. In a most preferred embodiment, the filamentous fungal host cell is an Aspergillus awamori, Aspergillus foetidus, Aspergillus japonicus, Aspergillus niger, Aspergillus nidulans or Aspergillus oryzae cell. In another most preferred embodiment, the filamentous fungal host cell is a Fusarium cell of the section Discolor (also known as the section Fusarium). For example, the filamentous fungal parent cell may be a Fusarium bactridioides, Fusarium cerealis, Fusarium crookwellense, Fusarium culmorum, Fusarium graminearum, Fusarium graminum, Fusarium heterosporum, Fusarium negundi, Fusarium reticulatum, Fusarium roseum, Fusarium sambucinum, Fusarium sarcochroum, Fusarium sulphureum, or Fusarium trichothecioides cell. In another prefered embodiment, the filamentous fungal parent cell is a Fusarium strain of the section Elegans, e.g., Fusarium oxysporum. In another most preferred embodiment, the filamentous fungal host cell is a Humicola insolens or Humicola lanuginosa cell. In another most preferred embodiment, the filamentous fungal host cell is a Mucor miehei cell. In another most preferred embodiment, the filamentous fungal host cell is a Myceliophthora thermophilum cell. In another most preferred embodiment, the filamentous fungal host cell is a Neurospora crassa cell. In another most preferred embodiment, the filamentous fungal host cell is a Penicillium purpurogenum cell. In another most preferred embodiment, the filamentous fungal host cell is a Thielavia terrestris cell or a Acremonium chrysogenum cell. In another most preferred embodiment, the Trichoderma cell is a Trichoderma harzianum, Trichoderma koningii, Trichoderma longibrachiatum, Trichoderma reesei or Trichoderma viride cell. The use of Aspergillus spp. for the expression of proteins is described in, e.g., EP 272 277, EP 230 023.

Transformation

Fungal cells may be transformed by a process involving protoplast formation, transformation of the protoplasts, and regeneration of the cell wall in a manner known per se. Suitable procedures for transformation of Aspergillus host cells are described in EP 238 023 and Yelton et al., 1984, Proceedings of the National Academy of Sciences USA 81:1470-1474. A suitable method of transforming Fusarium species is described by Malardier et al., 1989, Gene 78:147-156 or in copending U.S. Ser. No. 08/269,449. Examples of other fungal cells are cells of filamentous fungi, e.g. Aspergillus spp., Neurospora spp., Fusarium spp. or Trichoderma spp., in particular strains of A. oryzae, A. nidulans or A. niger. The use of Aspergillus spp. for the expression of proteins is described in, e.g., EP 272 277, EP 230 023, EP 184 . . . The transformation of F. oxysporum may, for instance, be carried out as described by Malardier et al., 1989, Gene 78:147-156.

Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153:163; and Hinnen et al., 1978, Proceedings of the National Academy of Sciences USA 75:1920. Mammalian cells may be transformed by direct uptake using the calcium phosphate precipitation method of Graham and Van der Eb (1978, Virology 52:546).

Transformation of insect cells and production of heterologous polypeptides therein may be performed as described in U.S. Pat. No. 4,745,051; No. 4,775,624; No. 4,879,236; No. 5,155,037; No. 5,162,222; EP 397,485) all of which are incorporated herein by reference. The insect cell line used as the host may suitably be a Lepidoptera cell line, such as Spodoptera frugiperda cells or Trichoplusia ni cells (cf. U.S. Pat. No. 5,077,214). Culture conditions may suitably be as described in, for instance, WO 89/01029 or WO 89/01028, or any of the aforementioned references.

Methods of Production

The transformed or transfected host cells described above are cultured in a suitable nutrient medium under conditions permitting the production of the desired molecules, after which these are recovered from the cells, or the culture broth.

The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The media are prepared using procedures known in the art (see, e.g., references for bacteria and yeast; Bennett, J. W. and LaSure, L., editors, More Gene Manipulations in Fungi, Academic Press, CA, 1991).

If the molecules are secreted into the nutrient medium, they can be recovered directly from the medium. If they are not secreted, they can be recovered from cell lysates. The molecules are recovered from the culture medium by conventional procedures including separating the host cells from the medium by centrifugation or filtration, precipitating the proteinaceous components of the supernatant or filtrate by means of a salt, e.g. ammonium sulphate, purification by a variety of chromatographic procedures, e.g. ion exchange chromatography, gelfiltration chromatography, affinity chromatography, or the like, dependent on the type of molecule in question.

The molecules of interest may be detected using methods known in the art that are specific for the molecules. These detection methods may include use of specific antibodies, formation of a product, or disappearance of a substrate. For example, an enzyme assay may be used to determine the activity of the molecule. Procedures for determining various kinds of activity are known in the art.

The molecules of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing (IEF), differential solubility (e.g., ammonium sulfate precipitation), or extraction (see, e.g., Protein Purification, J-C Janson and Lars Ryden, editors, VCH Publishers, New York, 1989).

The term “immunological response”, used in connection with the present invention, is the response of an organism to a compound, which involves the immune system according to any of the four standard reactions (Type I, II, III and IV according to Coombs & Gell).

Correspondingly, the “immunogenicity” of a compound used in connection with the present invention refers to the ability of this compound to induce an ‘immunological response’ in animals including man.

The term “allergic response”, used in connection with the present invention, is the response of an organism to a compound, which involves IgE mediated responses (Type I reaction according to Coombs & Gell). It is to be understood that sensibilization (i.e. development of compound-specific IgE antibodies) upon exposure to the compound is included in the definition of “allergic response”.

Correspondingly, the “allergenicity” of a compound used in connection with the present invention refers to the ability of this compound to induce an ‘allergic response’ in animals including man.

The terms “relevant protein backbone” or ‘protein backbone’ refer to the polypeptide to be modified by creating a library of diversified mutants. The “relevant protein backbone” may be a naturally occurring (or wild-type) polypeptide or it may be a variant thereof prepared by any suitable means. For instance, the “relevant protein backbone” may be a variant of a naturally occurring polypeptide which has been modified by substitution, deletion or truncation of one or more amino acid residues or by addition or insertion of one or more amino acid residues to the amino acid sequence of a naturally-occurring polypeptide.

The term “randomized library” of protein variants refers to a library with at least partially randomized composition of the members, e.g. protein variants.

The term “functionality” of protein variants refers to e.g. enzymatic activity, binding to a ligand or receptor, stimulation of a cellular response (e.g. ³H-thymidine incorporation as response to a mitogenic factor), or anti-microbial activity.

An “epitope” is a set of amino acids on a protein that are involved in an immunological response, such as antibody binding or T-cell activation. One particularly useful method of identifying epitopes involved in antibody binding is to screen a library of peptide-phage membrane protein fusions and selecting those that bind to relevant antigen-specific antibodies, sequencing the randomized part of the fusion gene, aligning the sequences involved in binding, defining consensus sequences based on these alignments, and mapping these consensus sequences on the surface or the sequence and/or structure of the antigen, to identify epitopes involved in antibody binding.

By the term “epitope pattern” is meant such a consensus sequence of peptides that bind well to a relevant antibody.

An “epitope area” is defined as the amino acids situated within 5 Å from the epitope amino acids. Modifications of amino acids of the ‘epitope area’ can possibly affect the function of the corresponding epitope.

By the term “specific polyclonal antibodies” is meant polyclonal antibodies isolated according to their specificity for a certain antigen, e.g. the protein backbone.

By the term “monospecific antibodies” is meant polyclonal antibodies isolated according to their specificity for a certain ‘epitope pattern’. Such monospecific antibodies will only bind to one epitope pattern, but they may very well be produced by a number of antibody producing cells and recognize the same epitope patterns, thereby being polyclonal.

‘Spiked mutagenesis’ is a form of site-directed mutagenesis, in which the primers used have been synthesized using mixtures of oligonucleotides at one or more positions.

### DETAILED DESCRIPTION OF THE INVENTION

The inventors have found a method for high throughput screening (HTS) of a large population of host cells for production of a molecule of interest.

By applying the present invention to a diversified library of protein variants it is possible to screen a large number of protein variants for their ability to bind to specific antibodies in a quick and automated manner thereby providing leads that may be tested for their immunogenicity in animal studies.

The present invention relates to method for screening a library of protein variants for functional variants with reduced antibody binding capacity, comprising the steps of:

(i) generating a diversified library of protein variants starting from a relevant protein backbone,

ii) transforming the library into suitable host cells,

iii) culturing host cells,

iv) sampling each cell culture,

v) analysing a sample by determining the antibody binding capacity of the variant protein,

vi) analysing a sample by determining the functionality of the variant protein.

Protein Backbone

The “relevant protein backbone” can in principle be any protein molecule of biological origin, non-limiting examples of which are peptides, polypeptides, proteins, enzymes, post-translationally modified polypeptides such as lipopeptides or glycosylated peptides, antimicrobial peptides or molecules, and proteins having pharmaceutical properties etc.

Accordingly the invention relates to a method, wherein the “relevant protein backbone” is chosen from the group consisting of polypeptides, small peptides, lipopeptides, antimicrobials, and pharmaceutical polypeptides.

The term “pharmaceutical polypeptides” is defined as polypeptides, including peptides, such as peptide hormones, proteins and/or enzymes, being physiologically active when administered to humans and/or animals.

Examples of “pharmaceutical polypeptides” contemplated according to the invention include insulin, ACTH, glucagon, somatostatin, somatotropin, thymosin, parathyroid hormone, pigmentary hormones, somatomedin, erythropoietin, luteinizing hormone, chorionic gonadotropin, hypothalmic releasing factors, antidiuretic hormones, thyroid stimulating hormone, relaxin, interferons, thrombopoietin (TPO), blood coagulation factors, plasminogen activators such as streptokinase and tissue-plasminogen activator, cerebrosidase, and prolactin.

However, the proteins are preferably to be used in industry, housekeeping and/or medicine, such as proteins used in personal care products (for example shampoo; soap; skin, hand and face lotions; skin, hand and face creams; hair dyes; toothpaste), food/feed (for example in the baking industry), detergents, anti-microbial compositions.

In one embodiment of the invention the protein is an enzyme, such as glycosyl hydrolases, carbohydrases, peroxidases, proteases, lipases, phytases, polysaccharide lyases, oxidoreductases, transglutaminases and glycoseisomerases, in particular the following.

Parent Proteases

Parent proteases (i.e. enzymes classified under the Enzyme Classification number E.C. 3.4 in accordance with the Recommendations (1992) of the International Union of Biochemistry and Molecular Biology (IUBMB)) include proteases within this group.

Examples include proteases selected from those classified under the Enzyme Classification (E.C.) numbers:

3.4.11 (i.e. so-called aminopeptidases), including 3.4.11.5 (Prolyl aminopeptidase), 3.4.11.9 (X-pro aminopeptidase), 3.4.11.10 (Bacterial leucyl aminopeptidase), 3.4.11.12 (Thermophilic aminopeptidase), 3.4.11.15 (Lysyl aminopeptidase), 3.4.11.17 (Tryptophanyl aminopeptidase), 3.4.11.18 (Methionyl aminopeptidase).

3.4.21 (i.e. so-called serine endopeptidases), including 3.4.21.1 (Chymotrypsin), 3.4.21.4 (Trypsin), 3.4.21.25 (Cucumisin), 3.4.21.32 (Brachyurin), 3.4.21.48 (Cerevisin) and 3.4.21.62 (Subtilisin);

3.4.22 (i.e. so-called cysteine endopeptidases), including 3.4.22.2 (Papain), 3.4.22.3 (Ficain), 3.4.22.6 (Chymopapain), 3.4.22.7 (Asclepain), 3.4.22.14 (Actinidain), 3.4.22.30 (Caricain) and 3.4.22.31 (Ananain);

3.4.23 (i.e. so-called aspartic endopeptidases), including 3.4.23.1 (Pepsin A), 3.4.23.18 (Aspergillopepsin I), 3.4.23.20 (Penicillopepsin) and 3.4.23.25 (Saccharopepsin); and

3.4.24 (i.e. so-called metalloendopeptidases), including 3.4.24.28 (Bacillolysin).

Examples of relevant subtilisins comprise subtilisin BPN′, subtilisin amylosacchariticus, subtilisin 168, subtilisin mesentericopeptidase, subtilisin Carlsberg, subtilisin DY, subtilisin 309, subtilisin 147, thermitase, aqualysin, Bacillus PB92 protease, proteinase K, Protease TW7, and Protease TW3.

Specific examples of such readily available commercial proteases include Esperase®, Alcalase®, Neutrase®, Dyrazym®, Savinase®, Pyrase®, Pancreatic Trypsin NOVO (PTN), Bio-Feed(Pro, Clear-Lens Pro (all enzymes available from Novo Nordisk A/S).

Examples of other commercial proteases include Maxtase®, Maxacal®, Maxapem® marketed by Gist-Brocades N. V., Opticlean® marketed by Solvay et Cie. and Purafect® marketed by

Genencor International.

It is to be understood that also protease variants are contemplated as the parent protease. Examples of such protease variants are disclosed in EP 130.756 (Genentech), EP 214.435 (Henkel), WO 87/04461 (Amgen), WO 87/05050 (Genex), EP 251.446 (Genencor), EP 260.105 (Genencor), Thomas et al., (1985), Nature. 318, p. 375-376, Thomas et al., (1987), J. Mol. Biol., 193, pp. 803-813, Russel et al., (1987), Nature, 328, p. 496-500, WO 88/08028 (Genex), WO 88/08033 (Amgen), WO 89/06279 (Novo Nordisk A/S), WO 91/00345 (Novo Nordisk A/S), EP 525 610 (Solvay) and WO 94/02618 (Gist-Brocades N. V.).

The activity of proteases can be determined as described in “Methods of Enzymatic Analysis”, third edition, 1984, Verlag Chemie, Weinheim, vol. 5.

Parent Lipases

Parent lipases (i.e. enzymes classified under the Enzyme Classification number E.C. 3.1.1 (Carboxylic Ester Hydrolases) in accordance with the Recommendations (1992) of the International Union of Biochemistry and Molecular Biology (IUBMB)) include lipases within this group.

Examples include lipases selected from those classified under the Enzyme Classification (E.C.) numbers:

3.1.1 (i.e. so-called Carboxylic Ester Hydrolases), including (3.1.1.3) Triacylglycerol lipases, (3.1.1.4.) Phosphorlipase A2.

Examples of lipases include lipases derived from the following microorganisms. The indicated patent publications are incorporated herein by reference:

Humicola, e.g. H. brevispora, H. lanuginosa, H. brevis var. thermoidea and H. insolens (U.S. Pat. No. 4,810,414).

Pseudomonas, e.g. Ps. fragi, Ps. stutzeri, Ps. cepacia and Ps. fluorescens (WO 89/04361), or Ps. plantarii or Ps. gladioli (U.S. Pat. No. 4,950,417 (Solvay enzymes)) or Ps. alcaligenes and Ps. pseudoalcaligenes (EP 218 272) or Ps. mendocina (WO 88/09367; U.S. Pat. No. 5,389,536).

Fusarium, e.g. F. oxysporum (EP 130,064) or F. solani pisi (WO 90/09446).

Mucor (also called Rhizomucor), e.g. M. miehei (EP 238 023). Chromobacterium (especially C. viscosum). Aspergillus (especially A. niger). Candida, e.g. C. cylindracea (also called C. rugosa) or C. antarctica (WO 88/02775) or C. antarctica lipase A or B (WO 94/01541 and WO 89/02916). Geotricum, e.g. G. candidum (Schimada et al., (1989), J. Biochem., 106, 383-388). Penicillium, e.g. P. camembertii (Yamaguchi et al., (1991), Gene 103,61-67). Rhizopus, e.g. R. delemar (Hass et al., (1991), Gene 109, 107-113) or R. niveus (Kugimiya et al., (1992) Biosci. Biotech. Biochem 56, 716-719) or R. oryzae. Bacillus, e.g. B. subtilis (Dartois et al., (1993)

Biochemica et Biophysica acta 1131, 253-260) or B. stearothermophilus (JP 64/7744992) or B. pumilus (WO 91/16422).

Specific examples of readily available commercial lipases include Lipolase®, Lipolase (Ultra, Lipozyme®, Palatase®, Novozym®435, Lecitase®(all available from Novo Nordisk A/S). Examples of other lipases are Lumafast (, Ps. mendocian lipase from Genencor Int. Inc.; Lipomax (, Ps. pseudoalcaligenes lipase from Gist Brocades/Genencor Int. Inc.; Fusarium solani lipase (cutinase) from Unilever; Bacillus sp. lipase from Solvay enzymes. Other lipases are available from other companies.

It is to be understood that also lipase variants are contemplated as the parent enzyme. Examples of such are described in e.g. WO 93/01285 and WO 95/22615.

The activity of the lipase can be determined as described in “Methods of Enzymatic Analysis”, Third Edition, 1984, Verlag Chemie, Weinhein, vol. 4, or as described in AF 95/5 GB (available on request from Novo Nordisk A/S).

Parent Oxidoreductases

Parent oxidoreductases (i.e. enzymes classified under the Enzyme Classification number E.C. 1 (Oxidoreductases) in accordance with the Recommendations (1992) of the International Union of Biochemistry and Molecular Biology (IUBMB)) include oxidoreductases within this group.

Examples include oxidoreductases selected from those classified under the Enzyme Classification (E.C.) numbers:

Glycerol-3-phosphate dehydrogenase _NAD+_ (1.1.1.8), Glycerol-3-phosphate dehydrogenase _NAD(P)+_ (1.1.1.94), Glycerol-3-phosphate 1-dehydrogenase _NADP_ (1.1.1.94), Glucose oxidase (1.1.3.4), Hexose oxidase (1.1.3.5), Catechol oxidase (1.1.3.14), Bilirubin oxidase (1.3.3.5), Alanine dehydrogenase (1.4.1.1), Glutamate dehydrogenase (1.4.1.2), Glutamate dehydrogenase _NAD(P)+_ (1.4.1.3), Glutamate dehydrogenase _NADP+_ (1.4.1.4), L-Amino acid dehydrogenase (1.4.1.5), Serine dehydrogenase (1.4.1.7), Valine dehydrogenase _NADP+_ (1.4.1.8), Leucine dehydrogenase (1.4.1.9), Glycine dehydrogenase (1.4.1.10), L-Amino-acid oxidase (1.4.3.2.), D-Amino-acid oxidase (1.4.3.3), L-Glutamate oxidase (1.4.3.11), Protein-lysine 6-oxidase (1.4.3.13), L-lysine oxidase (1.4.3.14), L-Aspartate oxidase (1.4.3.16), D-amino-acid dehydrogenase (1.4.99.1), Protein disulfide reductase (1.6.4.4), Thioredoxin reductase (1.6.4.5), Protein disulfide reductase (glutathione) (1.8.4.2), Laccase (1.10.3.2), Catalase (1.11.1.6), Peroxidase (1.11.1.7), Lipoxygenase (1.13.11.12), Superoxide dismutase (1.15.1.1)

Said Glucose oxidases may be derived from Aspergillus niger. Said Laccases may be derived from Polyporus pinsitus, Myceliophtora thermophila, Coprinus cinereus, Rhizoctonia solani, Rhizoctonia praticola, Scytalidium thermophilum and Rhus vemicifera. Bilirubin oxidases may be derived from Myrothechecium verrucaria. The Peroxidase may be derived from e.g. Soy bean, Horseradish or Coprinus cinereus. The Protein Disulfide reductase may be any of the mentioned in DK patent applications No. 768/93, 265/94 and 264/94 (Novo Nordisk A/S), which are hereby incorporated as references, including Protein Disulfide reductases of bovine origin, Protein Disulfide reductases derived from Aspergillus oryzae or Aspergillus niger, and DsbA or DsbC derived from Escherichia coli.

Specific examples of readily available commercial oxidoreductases include Gluzyme (enzyme available from Novo Nordisk A/S). However, other oxidoreductases are available from others. It is to be understood that also variants of oxidoreductases are contemplated as the parent enzyme.

The activity of oxidoreductases can be determined as described in “Methods of Enzymatic Analysis”, third edition, 1984, Verlag Chemie, Weinheim, vol. 3.

Parent Carbohydrases

Parent carbohydrases may be defined as all enzymes capable of breaking down carbohydrate chains (e.g. starches) of especially five and six member ring structures (i.e. enzymes classified under the Enzyme Classification number E.C. 3.2 (glycosidases) in accordance with the Recommendations (1992) of the International Union of Biochemistry and Molecular Biology (IUBMB)).

Examples include carbohydrases selected from those classified under the Enzyme Classification (E.C.) numbers:

(-amylase (3.2.1.1) (-amylase (3.2.1.2), glucan 1,4-(-glucosidase (3.2.1.3), cellulase (3.2.1.4), endo-1,3(4)-(-glucanase (3.2.1.6), endo-1,4-(-xylanase (3.2.1.8), dextranase (3.2.1.11), chitinase (3.2.1.14), polygalacturonase (3.2.1.15), lysozyme (3.2.1.17), (-glucosidase (3.2.1.21), (-galactosidase (3.2.1.22), (-galactosidase (3.2.1.23), amylo-1,6-glucosidase (3.2.1.33), xylan 1,4-(-xylosidase (3.2.1.37), glucan endo-1,3-(-D-glucosidase (3.2.1.39), (dextrin endo-1,6-glucosidase (3.2.1.41), sucrose (-glucosidase (3.2.1.48), glucan endo-1,3-(glucosidase (3.2.1.59), glucan 1,4-(-glucosidase (3.2.1.74), glucan endo-1,6-(-glucosidase (3.2.1.75), arabinan endo-1,5-(-arabinosidase (3.2.1.99), lactase (3.2.1.108), and chitonanase (3.2.1.132).

Examples of relevant carbohydrases include (-1,3-glucanases derived from Trichoderma harzianum; (-1,6-glucanases derived from a strain of Paecilomyces; (-glucanases derived from Bacillus subtilis; (-glucanases derived from Humicola insolens; (-glucan-ases derived from Aspergillus niger; (-glucanases derived from a strain of Trichoderma; (-glucanases derived from a strain of Oerskovia xanthineolytica; exo-1,4-(-D-glucosidases (glucoamylases) derived from Aspergillus niger; (-amylases derived from Bacillus subtilis; (-amylases derived from Bacillus amyloliquefaciens; (-amylases derived from Bacillus stearothermophilus; (-amylases derived from Aspergillus oryzae; (-amylases derived from non-pathogenic microorganisms; (galactosidases derived from Aspergillus niger; Pentosanases, xylanases, cellobiases, cellulases, hemi-cellulases deriver from Humicola insolens; cellulases derived from Trichoderma reesei; cellulases derived from non-pathogenic mold; pectinases, cellulases, arabinases, hemi-celluloses derived from Aspergillus niger; dextranases derived from Penicillium lilacinum; endo-glucanase derived from non-pathogenic mold; pullulanases derived from Bacillus acidopullyticus; (-galactosidases derived from Kluyveromyces fragilis; xylanases derived from Trichoderma reesei;

Specific examples of readily available commercial carbohydrases include Alpha-Gal(, Bio-Feed( Alpha, Bio-Feed(Beta, Bio-Feed(Plus, Bio-Feed(Plus, Novozyme® 188, Carezyme®, Celluclast®, Cellusoft®, Ceremyl®, Citrozym(, Denimax(, Dezyme(, Dextrozyme(, Finizym®, Fungamyl(, Gamanase(, Glucanex(®, Lactozym®, Maltogenase(, Pentopan(, Pectinex(, Promozyme®, Pulpzyme(, Novamyl(, Termamyl®, AMG (Amyloglucosidase Novo), Maltogenase®, Aquazym®, Natalase( (all enzymes available from Novo Nordisk A/S). Other carbohydrases are available from other companies.

It is to be understood that also carbohydrase variants are contemplated as the parent enzyme. The activity of carbohydrases can be determined as described in “Methods of Enzymatic Analysis”, third edition, 1984, Verlag Chemie, Weinheim, vol. 4.

Parent Transferases

Parent transferases (i.e. enzymes classified under the Enzyme Classification number E.C. 2 in accordance with the Recommendations (1992) of the International Union of Biochemistry and Molecular Biology (IUBMB)) include transferases within this group.

The parent transferases may be any transferase in the subgroups of transferases: transferases transferring one-carbon groups (E.C. 2.1); transferases transferring aldehyde or residues (E.C 2.2); acyltransferases (E.C. 2.3); glucosyltransferases (E.C. 2.4); transferases transferring alkyl or aryl groups, other that methyl groups (E.C. 2.5); transferases transferring nitrogeneous groups (2.6).

In a preferred embodiment the parent transferase is a transglutaminase E.C 2.3.2.13(Proteinglutamine (-glutamyltransferase).

Transglutaminases are enzymes capable of catalyzing an acyl transfer reaction in which a gamma-carboxyamide group of a peptide-bound glutamine residue is the acyl donor. Primary amino groups in a variety of compounds may function as acyl acceptors with the subsequent formation of monosubstituted gamma-amides of peptide-bound glutamic acid. When the epsilon-amino group of a lysine residue in a peptide-chain serves as the acyl acceptor, the transferases form intramolecular or intermolecular gamma-glutamyl-epsilon-lysyl crosslinks. Examples of transglutaminases are described in the pending DK patent application no. 990/94 (Novo Nordisk A/S).

The parent transglutaminase may be of human, animal (e.g. bovine) or microbial origin. Examples of such parent transglutaminases are animal derived Transglutaminase, FXIIIa; microbial transglutaminases derived from Physarum polycephalum (Klein et al., Journal of Bacteriology, Vol. 174, p. 2599-2605); transglutaminases derived from Streptomyces sp., including Streptomyces lavendulae, Streptomyces lydicus (former Streptomyces libani) and Streptoverticillium sp., including Streptoverticillium mobaraense, Streptoverticillium cinnamoneum, and Streptoverticillium griseocarneum (Motoki et al., U.S. Pat. No. 5,156,956; Andou et al., U.S. Pat. No. 5,252,469; Kaempfer et al., Journal of General Microbiology, Vol. 137, p. 1831-1892; Ochi et al., International Journal of Sytematic Bacteriology, Vol. 44, p. 285-292; Andou et al., U.S. Pat. No. 5,252,469; Williams et al., Journal of General Microbiology, Vol. 129, p. 1743-1813). It is to be understood that also transferase variants are contemplated as the parent enzyme. The activity of transglutaminases can be determined as described in “Methods of Enzymatic Analysis”, third edition, 1984, Verlag Chemie, Weinheim, vol. 1-10.

Parent Phytases

Parent phytases are included in the group of enzymes classified under the Enzyme Classification number E.C. 3.1.3 (Phosphoric Monoester Hydrolases) in accordance with the Recommendations (1992) of the International Union of Biochemistry and Molecular Biology (IUBMB)).

Phytases are enzymes produced by microorganisms, which catalyse the conversion of phytate to inositol and inorganic phosphorus.

Phytase producing microorganisms comprise bacteria such as Bacillus subtilis, Bacillus natto and Pseudomonas; yeasts such as Saccharomyces cerevisiae; and fungi such as Aspergillus niger, Aspergillus ficuum, Aspergillus awamori, Aspergillus oryzae, Aspergillus terreus or Aspergillus nidulans, and various other Aspergillus species).

Examples of parent phytases include phytases selected from those classified under the Enzyme Classification (E.C.) numbers: 3-phytase (3.1.3.8) and 6-phytase (3.1.3.26).

The activity of phytases can be determined as described in “Methods of Enzymatic Analysis”, third edition, 1984, Verlag Chemie, Weinheim, vol. 1-10, or may be measured according to the method described in EP-A1-0 420 358, Example 2 A.

Lyases

Suitable lyases include Polysaccharide lyases: Pectate lyases (4.2.2.2) and pectin lyases (4.2.2.10), such as those from Bacillus licheniformis disclosed in WO 99/27083.

Isomerases:

Protein Disulfide Isomerase.

Without being limited thereto suitable protein disulfide isomerases include PDIs described in WO 95/01425 (Novo Nordisk A/S) and suitable glucose isomerases include those described in Biotechnology Letter, Vol. 20, No 6, June 1998, pp. 553-56.

Contemplated isomerases include xylose/glucose Isomerase (5.3.1.5) including Sweetzyme®.

Identifying Areas of Interest for Introduction of Modifications

The methods of this invention are especially suitable when testing compounds that are being modified with respect to their allergenicity.

Such modification of a test compound to affect its immunogenecity could be by mutation of a protein allergen in its immunoglobulin-specific epitopes. The location of these epitopes can be determined by several techniques such as those disclosed by WO 92/10755 (by U. Løvborg), by Walshet et al, J. Immunol. Methods, vol. 121, 1275-280, (1989), and by Schoofs et al. J. Immunol. vol. 140, 611-616, (1987). A preferred method for identification of epitopes is by screening a random peptide library with antibodies (e.g. IgE or IgG antibodies), selecting high-binding peptides, obtaining the sequence these, and aligning the high-binding peptide sequences to identify a consensus sequence. These consensus sequences, in turn are compared with the sequence and 3D structure of a relevant protein, which is desired to mutate for reduction of immunogenicity, in order to identify the linear and structural epitopes of that protein.

In an even more preferred method, the identification of epitope(s) may be achieved by screening of phage display libraries. The principle behind phage display is that a heterologous DNA sequence can be inserted in the gene coding for a coat protein of the phage. The phage will make and display the hybrid protein on its surface where it can interact with specific target agents. Such target agent may be antigen-specific antibodies. It is therefore possible to select specific phages that display antibody-binding peptide sequences. The displayed peptides can be of predetermined lengths, for example 9 amino acids long, with randomized sequences, resulting in a random peptide display package library. Thus, by screening for antibody binding, one can isolate the peptide sequences that have the highest affinity for the particular antibody used. The peptides of the hybrid proteins of the specific phages which bind protein-specific antibodies define the epitopes of that particular protein. The corresponding residues of the parent protein can be found by aligning the selected peptide sequences resulting in epitope patterns, and compare these with the amino acid sequence and 3-dimensional structure of the parent protein.

When the epitope(s) have been identified, a protein variant exhibiting a reduced immunogenicity may be produced by changing the identified epitope pattern of the parent protein by genetic engineering of a DNA sequence encoding the parent protein. It is commonly found, that amino acids surrounding B- and T-cell epitopes can affect binding of the antibodies or T-cell receptors to the antigen. To anticipate this possibility, an epitope area was defined on the 3-dimensional structure of the protein of interest, and genetic engineering of any amino acid within the epitope area is considered to be within the scope of using the identified epitope to generate variants with low immunogenecity.

Generating a Diversified Library

In order to generate protein variants, more than one amino acid residue may be substituted, added or deleted, these amino acids preferably being located in different epitope areas. In that case, it may be difficult to assess a priori how well the functionality of the protein is maintained while antigenicity is reduced, especially since the possible number of combinations of mutations become very large, even for a small number of mutations. In that case, it will be an advantage, to establish a library of diversified mutants each having one or more changed amino acids introduced and selecting those variants, which show good retention of function and at the same time a significant reduction in antigenicity.

A diversified library can be established by a range of techniques known to the person skilled in the art (Reetz M T; Jaeger K E, in ‘Biocatalysis—from Discovery to Application’ edited by Fessner WD, Vol. 200, pp. 31-57 (1999); Stemmer, Nature, vol. 370, p.389-391, 1994; Zhao and Arnold, Proc. Natl. Acad. Sci., USA, vol. 94, pp. 7997-8000, 1997; or Yano et al., Proc. Natl. Acad. Sci., USA, vol. 95, pp 5511-5515, 1998).

These include, but are not limited to, ‘spiked mutagenesis’, in which certain positions of the protein sequence are randomized by carring out PCR mutagenesis using one or more oligonucleotide primers which are synthesized using a mixture of nucleotides for certain positions (Lanio T, Jeltsch A, Biotechniques, Vol. 25(6), 958,962,964-965 (1998)). The mixtures of oligonucleotides used within each triplet can be designed such that the corresponding amino acid of the mutated gene product is randomized within some predetermined distribution function. Algorithms exist, which facilitate this design (Jensen L J et al., Nucleic Acids Research, Vol. 26(3), 697-702 (1998)).

In an embodiment substitutions are found by a method comprising the following steps: 1) a range of substitutions, additions, and/or deletions are listed encompassing several epitope areas, 2) a library is designed which introduces a randomized subset of these changes in the amino acid sequence into the target gene, e.g. by spiked mutagenesis, 3) the library is expressed, and preferred variants are selected. In another embodiment, this method is supplemented with additional rounds of screening and/or family shuffling of hits from the first round of screening (J. E. Ness, et al, Nature Biotechnology, vol. 17, pp. 893-896, 1999) and/or combination with other methods of reducing immunogenicity by genetic means (such as that disclosed in WO92/10755).

The library may be designed, such that at least one amino acid of the epitope area is substituted. In a preferred embodiment at least one amino acid of the epitope itself is changed. The library may be biased such that towards introducing an amino acid of different size, hydrophilicity, and/or polarity relative to the original one of the ‘protein backbone’. For example changing a small amino acid to a large amino acid, a hydrophilic amino acid to a hydrophobic amino acid, a polar amino acid to a non-polar amino acid or a basic to an acidic amino acid. Other changes may be the addition or deletion of at least one amino acid of the epitope area, preferably deleting an anchor amino acid. Furthermore, substituting some amino acids and deleting or adding others may change an epitope.

In another embodiment, the library is designed, such that recognition sites for post-translational modifications are introduced in the epitope areas, and the library is expressed in a suitable host organism capable of the corresponding post-translational modification. These post-translational modifications may serve to shield the epitope and hence lower the immunogenicity of the protein variant relative to the protein backbone. Post-translational modifications include glycosylation, phosphorylation, N-terminal processing, acylation, ribosylation and sulfatation. A good example is N-glycosylation. N-glycosylation is found at sites of the sequence Asn-Xaa-Ser, Asn-Xaa-Thr, or Asn-Xaa-Cys, in which neither the Xaa residue nor the amino acid following the tri-peptide consensus sequence is a proline (T. E. Creighton, ‘Proteins—Structures and Molecular Properties, 2nd edition, W.H. Freeman and Co., New York, 1993, pp. 91-93). It is thus desirable to introduce such recognition sites in the sequence of the backbone protein. The specific nature of the glycosyl chain of the glycosylated protein variant may be linear or branched depending on the protein and the host cells. Another example is phosphorylation: The protein sequence can be modified so as to introduce serine phophorylation sites with the recognition sequence arg-arg-(xaa)ₙ-ser (where n=0, 1, or 2), which can be phosphorylated by the cAMP-dependent kinase or tyrosine phosphorylation sites with the recognition sequence-lys/arg-(xaa)₃-asp/glu-(xaa)₃-tyr, which can usually be phophorylated by tyrosine-specific kinases (T. E. Creighton, “Protein-Structures and molecular properties”, 2nd ed., Freeman, N.Y., 1993).

Covalent Conjugation to Amino Acids in the Epitope Area.

In yet another embodiment, one can design the libarary, such that amino acids suitable for chemical modification are substituted for existing ones in the epitope areas. The protein variant can then be conjugated to activated polymers Which amino acids to substitute and/or insert depends in principle on the coupling chemistry to be applied. The chemistry for preparation of covalent bioconjugates can be found in “Bioconjugate Techniques”, Hermanson, G. T. (1996), Academic Press Inc., which is hereby incorporated as reference (see below). It is preferred to make conservative substitutions in the polypeptide when the polypeptide has to be conjugated, as conservative substitutions secure that the impact of the substitution on the polypeptide structure is limited. In the case of providing additional amino groups this may be done by substitution of arginine to lysine, both residues being positively charged, but only the lysine having a free amino group suitable as an attachment groups. In the case of providing additional carboxylic acid groups the conservative substitution may for instance be an asparagine to aspartic acid or glutamine to glutamic acid substitution. These residues resemble each other in size and shape, except from the carboxylic groups being present on the acidic residues. In the case of providing SH-groups the conservative substitution may be done by changing threonine or serine to cysteine.

Diversity in the protein variant library can be generated at the DNA triplet level, such that individual codons are variegated e.g. by using primers of partially randomized sequence for a PCR reaction. Further, several techniques have been described, by which one can create a library with such diversity at several locations in the gene, which are too far apart to be covered by a single (spiked) oligonucleotide primer. These techniques include the use of in vivo recombination of the individually diversified gene segments as described in WO 97/07205 on page 3, line 8 to 29 or by using DNA shuffling techniques to create a library of full length genes that combine several gene segments each of which are diversified e.g. by spiked mutagenesis (Stemmer, Nature 370, pp. 389-391, 1994 and U.S. Pat. Nos. 5,605,793 and 5,830,721). In the latter case, one can use the gene encoding the “protein backbone” as a template double-stranded polynucleotide and combining this with one or more single or double-stranded oligonucleotides as described in claim 1 of U.S. Pat. No. 5,830,721. The single-stranded oligonucleotides could be partially randomized during synthesis. The double-stranded oligonucleotides could be PCR products incorporating diversity in a specific region. In both cases, one can dilute the diversity with corresponding segments containing the sequence of the backbone protein in order to limit the number of changes that are on average introduced. As mentioned above, methods have been established for designing the ratios of nucleotides (A; C; T; G) used at a particular codon during primer synthesis, so as to approximate a desired frequency distribution among a set of desired amino acids at that particular codon. This allows one to bias the partially randomized mutagenesis towards e.g. introduction of post-translational modification sites, chemical modification sites, or simply amino acids that are different from those that define the epitope or the epitope area. One could also approximate a sequence in a given location or epitope area to the corresponding location on a homologous, human protein.

When one uses protein engineering to eliminate epitopes, it is indeed possible that new epitopes are created, or existing epitopes are duplicated. To reduce this risk, one can map the planned mutations at a given position on the 3-dimensional structure of the protein of interest, and control the emerging amino acid constellation against a database of known epitope patterns, to rule out those possible replacement amino acids, which are predicted to result in creation or duplication of epitopes. Thus, risk mutations can be identified and eliminated by this procedure, thereby reducing the number of mutations at each position, and hence reducing the library size.

Occasionally, one would be interested in testing a library that combines a number of known mutations in different locations in the primary sequence of the ‘protein backbone’. These could be introduced post-translational or chemical modification sites, or they could be mutations, which by themselves had proven beneficial for one reason or another (e.g. decreasing antigenicity, or improving specific activity, performance, stability, or other characteristics). In such cases, it may be desirable to create a library of diverse combinations of known sequences. For example if 12 individual mutations are known, one could combine (at least) 12 segments of the ‘protein backbone’ gene in which each segment is present in two forms: one with and one without the desired mutation. By varying the relative amounts of those segments, one could design a library (of size 2¹²) for which the average number of mutations per gene can be predicted. This can be a useful way of combining elements that by themselves give some, but not sufficient effect, without resorting to very large libraries, as is often the case when using ‘spiked mutagenesis’.

Host Cells, Culturing, and Sampling

As described above, any number of host cells can be used to perform the method of the invention. Preferably the host cells are of microbial origin, preferably bacterial, yeast, or fungal. Even more preferably the host cells are chosen from the group consisting of Escherichia coli, Bacillus subtilis, Bacillus licheniformis, Bacillus clausii, Aspergillus niger, Aspergillus oryzae, Aspergillus nidulans, and Saccharomyces cerevisiae.

The diversified library is prepared as a DNA library of genes encoding variants of the relevant protein backbone. This DNA library is then transformed into the host cells by using any of the techniques known in the art and described above. Typically, one would want to culture the cells in different positions of a spatial array, necessitating the distribution of individual clones into each position of the array. This is ideally done in such a way that each position is occupied by exactly one cell. In practice, however, the number of cells at each position will follow a probability distribution. Hence, in a preferred embodiment, the average number of cells per well is between 0, 2 and 1 cell. In a more preferred embodiment, the number of cells per well is optimized such that the highest density of array positions occupied by exactly one cell is obtained. The number of cells at each position can be controlled by dilution. The dilution that most closely approximates one cell per position is often termed ‘the limiting dilution’.

Steps (iii) through (vi) of the present invention can be performed in many ways. Typically, the culturing and the sampling of host cells takes place using a spatial array. The spatial array can take on any physical form whatsoever, that enables the culturing or assaying of several samples at once, without one sample contaminating another. Examples of preferred spatial arrays are different kinds of microtiter-plates with any number of wells, such as 96 or 384, and of any kind of material, as well as positions in a High Performance Liquid Chromatography (HPLC) autosampler device. Any kind of physical arrangement which allows the unambiguous identification of the samples by a number or a position in the array. Even samples placed as drops on a surface in a specific recorded pattern, the surface being of a solid material or of more complex nature such as a textile or a tissue, e.g. cotton, wool, paper, or cellulose.

A preferred embodiment relates to a method, wherein the spatial array of is a microtiter plate, a solid surface or a textile surface.

A way of carrying out step (iv) of the first aspect of the invention could be to take a sample from each position of the spatial array, e.g. from a supernatant or cell culture, and transfer this to another spatial array for further testing or assaying for production of the molecule of interest. The second spatial array may be identical to the first one used in the specific method, but may also be of any other kind that fulfills the above mentioned criteria, such as a microtiter plate, a solid surface, a textile, any material etc.

Accordingly a preferred embodiment relates to a method, wherein after step (iv) a sample is transferred from each position in the spatial array to a position in a second spatial array which is then used onwards in the method, preferably the second array is a microtiter plate.

In one aspect of the current invention, the individual cells/clones are grown within microenvironments in each position of the spatial array. Such microenvironments are initally sterile beads or balls of any material that allows growth of the clone, preferably beads comprising agarose, alginate, polysaccharide, carbohydrate, alginate, carrageenan, chitosan, cellulose, pectin, dextran or polyacrylamide, all allowing diffusion to/from each microenvironment.

Accordingly a preferred embodiment relates to a method of the first aspect, wherein each position in the spatial array is occupied by a bead comprising one cell, preferably the bead is an agarose-bead.

The assaying of the invention for production of the molecule of interest can be done in a great number of ways. As indicated, some kinds of spatial arrays like microtiter plates, can sometimes be assayed directly, or samples can be taken from each position and tranferred to another spatial array for assaying according to step (v) and (vi) or according to any of a number of techniques well known in the art, such as enzyme activity, receptor binding, and many others; common to these assays is that a measurement is taken of a detectable property e.g. fluorescence, luminescence, absorption. The method of the invention can be performed with any number of these assays, and consequently the molecules assayed for in step (v) and (vi) can be obtained in a number of ways, depending on the host cell construct. The molecule of interest may be secreted by the host cell into the supernatant, or the molecule may remain intracellular, in which case lysis of the host cells may release the molecule.

A preferred embodiment relates to a method, wherein the molecule of interest in (v) and (vi) is assayed in either whole broth, supernatant of cells that secrete the molecule, a lysate of cells that produce the molecule, or is assayed while still inside cells that produce the molecule.

In another embodiment, the sample is purified by a size separation process, such as the membrane processes filtration and dialysis, prior to the analyses of step (v) and (vi). This will serve to reduce interference from particulate matter or high molecular mass compounds from the cells (which are larger than the soluble protein variants) or from the small molecule metabolites, substrate compounds, or degradation products (which are smaller than the protein variants). This size separation can be achieved, e.g. by using microtiter well inserts (e.g. Nunc TC), vacuum filtration microtiter plates (e.g. Qiagen, QIAwell) or pumping or centrifugation devices.

Extra Steps

In one aspect of the present invention the host cells may be first selected based on a functional screen, and then re-cultured, sampled, and analysed for antibody binding capacity and functionality. The initial functional selection can be done using an agar plate assay to analyse colonies from the transformed cells and select for those resulting in halo formation (see for example Ness, J. E:, et al., Nature Biotechn., 17, pp 893-896, 1999). Then, host cells expressing functional protein variants may be selected, preferably by automated colony picking, before the antibody binding capacity of the protein variant is assayed. This mode can increase the throughput and quality of the screening setup in cases where the antibody binding capacity assay is slower, more cumbersome, more expensive, or less accurate than the functionality assay.

In another embodiment of the invention, protein variant is exposed to adverse conditions prior to analyzing the functionality, in order to gauge also the stability of the protein variant. Alternatively, the functionality is determined twice with the protein variant being exposed to adverse conditions for a period of time in between the two determinations of functionality. From these measurements the stability of the protein variant at the particular set of adverse conditions can be determined. These adverse conditions could be characterized by increased temperature, increased or decreased pH, the presence of certain metal ions or of metal chelators such as EDTA. They could also be the presence of surfactant molecules, such as those used in detergents, skin cream formulations, hand dish washing compositions or other applications; the presence of proteases or other degradative enzymes; or they could be the presence of enzyme inhibitors. In this embodiment, one records the stability as an additional parameter, which may help in selecting the protein variants for further culturing and analysis.

In another embodiment, the protein variant of the sample (step iv) is immobilized to a solid material. This will be an advantage, for instance by facilitating removal of impurities, chemical modification of the protein variant, and assessment of the antibody binding capacity of the protein variant. The solid material could be the well surface of a microtiterplate, suspended beads, the pin of dipstick devices or others. Immobilization can be either non-specific by hydrophobic interactions, ionic interactions, or chemical coupling, or it can be more specific, such as non-covalent coupling to immobilized binding partners such as antibodies, enzyme inhibitors, or substrate mimics. In a preferred embodiment, the protein backbone has been modified genetically with an N-terminal or C-terminal extension comprising a high affinity tag for a compound, which can be immobilized. An example is a polyhistidine tag, which binds with high affinity to Nickel, which in turn has been bound to an acid such as nitrilotriacetic acid which has been immobilized to the solid phase. Other examples of an affinity tags are the cellulose binding domains of bacteria and fungi, which bind with high affinity to cellulose (such as Avicell), also when fused onto heterogeneous proteins, calmodulin binding domains, S-tag or FLAG peptides that bind to specific antibodies etc.

In a preferred embodiment, the binding to the solid phase is reversible, such that the protein variant can be eluted into solution when exposed to certain conditions such as e.g. high salt concentrations, high pH, or high competitor concentration.

In another embodiment, efficient HTS is achieved by devicing a simple yet accurate determination of the amount of a specific active molecule produced by the individual clone. One solution is that when the concentration of the specific molecule in a position of the spatial array has been determined, this information can be used to determine the specific activity from the total activity determined in that position; alternatively, the information can be used to adjust the input of the molecule into the activity assay. A second solution is to use immobilization of the protein variant as a means to dose the subsequent assays with a known and/or constant amount of protein variant. This requires that the sample contain more protein variant than the binding capacity of the immobilization step. Alternatively, the assay can be configured in such a way that it is insensitive to the concentration of the molecule.

Chemical Conjugation

In one embodiment, the protein variants are being modified by chemical conjugation prior to the analyses of step (v) and (vi). For this, the protein variant needs to be incubate with an active or activated polymer and subsequently separated from the unreacted polymer. This can conveniently be done using the immobilized protein variants, which can easily be exposed to different reaction environments and washes.

In the case were polymeric molecules are to be conjugated with the polypeptide in question and the polymeric molecules are not active they must be activated by the use of a suitable technique. It is also contemplated according to the invention to couple the polymeric molecules to the polypeptide through a linker. Suitable linkers are well-known to the skilled person.

Methods and chemistry for activation of polymeric molecules as well as for conjugation of polypeptides are intensively described in the literature. Commonly used methods for activation of insoluble polymers include activation of functional groups with cyanogen bromide, periodate, glutaraldehyde, biepoxides, epichlorohydrin, divinylsulfone, carbodiimide, sulfonyl halides, trichlorotriazine etc. (see R. F. Taylor, (1991), “Protein immobilisation. Fundamental and applications”, Marcel Dekker, N.Y.; S. S. Wong, (1992), “Chemistry of Protein Conjugation and Crosslinking”, CRC Press, Boca Raton; G. T. Hermanson et al., (1993), “Immobilized Affinity Ligand Techniques”, Academic Press, N.Y.). Some of the methods concern activation of insoluble polymers but are also applicable to activation of soluble polymers e.g. periodate, trichlorotriazine, sulfonylhalides, divinylsulfone, carbodiimide etc. The functional groups being amino, hydroxyl, thiol, carboxyl, aldehyde or sulfydryl on the polymer and the chosen attachment group on the protein must be considered in choosing the activation and conjugation chemistry which normally consist of i) activation of polymer, ii) conjugation, and iii) blocking of residual active groups.

In the following a number of suitable polymer activation methods will be described shortly. However, it is to be understood that also other methods may be used.

Coupling polymeric molecules to the free acid groups of polypeptides may be performed with the aid of diimide and for example amino-PEG or hydrazino-PEG (Pollak et al., (1976), J. Am. Chem. Soc., 98, 289-291) or diazoacetate/amide (Wong et al., (1992), “Chemistry of Protein Conjugation and Crosslinking”, CRC Press).

Coupling polymeric molecules to hydroxy groups is generally very difficult as it must be performed in water. Usually hydrolysis predominates over reaction with hydroxyl groups.

Coupling polymeric molecules to free sulfhydryl groups can be achieved with special groups like maleimido or the ortho-pyridyl disulfide. Also vinylsulfone (U.S. Pat. No. 5,414,135, (1995), Snow et al.) has a preference for sulfhydryl groups but is not as selective as the other mentioned.

Accessible Arginine residues in the polypeptide chain may be targeted by groups comprising two vicinal carbonyl groups.

Techniques involving coupling of electrophilically activated PEGs to the amino groups of Lysines may also be useful. Many of the usual leaving groups for alcohols give rise to an amine linkage. For instance, alkyl sulfonates, such as tresylates (Nilsson et al., (1984), Methods in Enzymology vol. 104, Jacoby, W. B., Ed., Academic Press: Orlando, p. 56-66; Nilsson et al., (1987), Methods in Enzymology vol. 135; Mosbach, K., Ed.; Academic Press: Orlando, pp. 65-79; Scouten et al., (1987), Methods in Enzymology vol. 135, Mosbach, K., Ed., Academic Press: Orlando, 1987; pp 79-84; Crossland et al., (1971), J. Amr. Chem. Soc. 1971, 93, pp. 4217-4219), mesylates (Harris, (1985), supra; Harris et al., (1984), J. Polym. Sci. Polym. Chem. Ed. 22, pp 341-352), aryl sulfonates like tosylates, and para-nitrobenzene sulfonates can be used.

Organic sulfonyl chlorides, e.g. Tresyl chloride, effectively converts hydroxy groups in a number of polymers, e.g. PEG, into good leaving groups (sulfonates) that, when reacted with nucleophiles like amino groups in polypeptides allow stable linkages to be formed between polymer and polypeptide. In addition to high conjugation yields, the reaction conditions are in general mild (neutral or slightly alkaline pH, to avoid denaturation and little or no disruption of activity), and satisfy the non-destructive requirements to the polypeptide.

Tosylate is more reactive than the mesylate but also less stable decomposing into PEG, dioxane, and sulfonic acid (Zalipsky, (1995), Bioconjugate Chem., 6, 150-165). Epoxides may also been used for creating amine bonds but are much less reactive than the abovementioned groups.

Converting PEG into a chloroformate with phosgene gives rise to carbamate linkages to Lysines. Essentially the same reaction can be carried out in many variants substituting the chlorine with N-hydroxy succinimide (U.S. Pat. No. 5,122,614, (1992); Zalipsky et al., (1992), Biotechnol. Appl. Biochem., 15, p. 100-114; Monfardini et al., (1995), Bioconjugate Chem., 6, 62-69, with imidazole (Allen et al., (1991), Carbohydr. Res., 213, pp 309-319), with para-nitrophenol, DMAP (EP 632 082 A1, (1993), Looze, Y.) etc. The derivatives are usually made by reacting the chloroformate with the desired leaving group. All these groups give rise to carbamate linkages to the peptide.

Furthermore, isocyanates and isothiocyanates may be employed, yielding ureas and thioureas, respectively.

Amides may be obtained from PEG acids using the same leaving groups as mentioned above and cyclic imid thrones (U.S. Pat. No. 5,349,001, (1994), Greenwald et al.). The reactivity of these compounds are very high but may make the hydrolysis to fast.

PEG succinate made from reaction with succinic anhydride can also be used. The hereby comprised ester group make the conjugate much more susceptible to hydrolysis (U.S. Pat. No. 5,122,614, (1992), Zalipsky). This group may be activated with N-hydroxy succinimide. Furthermore, a special linker can be introduced. The most well studied being cyanuric chloride (Abuchowski et al., (1977), J. Biol. Chem., 252, 3578-3581; U.S. Pat. No. 4,179,337, (1979), Davis et al.; Shafer et al., (1986), J. Polym. Sci. Polym. Chem. Ed., 24, 375-378.

Coupling of PEG to an aromatic amine followed by diazotation yields a very reactive diazonium salt, which can be reacted with a peptide in situ. An amide linkage may also be obtained by reacting an azlactone derivative of PEG (U.S. Pat. No. 5,321,095, (1994), Greenwald, R. B.) thus introducing an additional amide linkage.

As some peptides do not comprise many Lysines it may be advantageous to attach more than one PEG to the same Lysine. This can be done e.g. by the use of 1,3-diamino-2-propanol. PEGs may also be attached to the amino-groups of the enzyme with carbamate linkages (WO 95/11924, Greenwald et al.). Lysine residues may also be used as the backbone.

The coupling technique used in the examples is the N-succinimidyl carbonate conjugation technique descried in WO 90/13590 (Enzon).

In a preferred embodiment, the activated polymer is methyl-PEG which has been activated by N-succinimidyl carbonate as described WO 90/13590. The coupling can be carried out at alkaline conditions in high yields.

For coupling of polymers to the protein variants, it is preferred to use conditions similar to those described in WO96/17929 and WO99/00489 (Novo Nordisk A/S) e.g. mono or bis activated PEG's of molecular weight ranging from 100 to 5000 Da. For instance, a methyl-PEG 350 could be activated with N-succinimidyl carbonate and incubated with protein variant at a molar ratio of more than 5 calculated as equivalents of activated PEG divided by moles of lysines in the protein of interest. For coupling to immobilized protein variant, the PEG:protein ratio should be optimized such that the PEG concentration is low enough for the buffer capacity to maintain alkaline pH throughout the reaction; while the PEG concentration is still high enough to ensure sufficient degree of modification of the protein. Further, it is important that the activated PEG is kept at conditions that prevent hydrolysis (i.e. dissolved in acid or solvents) and diluted directly into the alkaline reaction buffer. It is essential that primary amines are not present other than those occurring in the lysine residues of the protein. This can be secured by washing thoroughly in borate buffer. The reaction is stopped by separating the fluid phase containing unreacted PEG from the solid phase containing protein and derivatized protein. Optionally, the solid phase can then be washed with tris buffer, to block any unreacted sites on PEG chains that might still be present.

Determining Antibody Binding Capacity

In step (v) of the first aspect of the invention, a sample is analysed to determine the antibody binding capacity of its variant protein. The antibodies used for this analysis can be in the form of serum isolated from an animal such as rabbit, mouse, rat, guinea pig, sheep etc., which has previously been exposed to the protein backbone. Optionally, the serum can be human serum from a volunteer who is known to have a history of exposure to the protein backbone. Preferably, serum samples are pooled from several animal or human donors to achieve an individual-independent result of the screening. Alternatively, the serum can be purified (e.g. by caprylic acid precipitation and DEAE chromatography) to achieve an immunoglobulin G fraction, or the serum can be purified using a Protein A column and/or an affinity column with anti-IgE (Fcε) antibodies, to prepare an IgE fraction. These and other methods of antibody purification are described in Harlow and Lane, “Antibodies—A laboratory manual”, Cold Spring Harbor Laboratory, 1988 and in Catty and Raykundalia: Production and Quality Control of Polyclonal Antibodies in “Antibodies—a practical approach” vol. 1, IRL Press, Oxford, 1988.

When the objective of using the HTS method of this invention is to reduce allergenicity of the protein variants, the IgE-purification method is preferred, and even more preferable is an embodiment in which the experimental animal has been intratracheally exposed to the protein backbone and has been shown to develop antigen-specific IgE antibodies, or if the human volunteers have a history of allergenic sensitisation to the protein backbone.

Whether an IgG, IgE, or other immunoglobulin fraction is used, it is preferable to increase the specificity of the antibody binding analysis by further purification. This can be in the form of affinity purification using a solid phase of immobilized antigen (e.g. the protein backbone). Immobilization can be by chemical conjugation, specific binding to a ligand or an antibody, or by binding through a fusion tag such as a polyhistidine tage, a FLAG peptide or the like. Antibodies are bound and eluted (e.g. using 1 M propionic acid) resulting in a “specific polyclonal antibody preparation” (see Arvieuz and Williams: Immunoaffinity Chromatography in “Antibodies—a practical approach” vol. 1, IRL Press, Oxford, 1988). In the case of using a protease antigen it may be desirable to reduce or eliminate protease activity during the antibody purification step. This can be done by several methods, including binding to an immobilized inhibitor (such as bacitracin in the case of subtilisins), using a chemically inactivated protease backbone (e.g. by PMSF treatment), or by using a mutated protease (e.g. by converting the catalytically active serine to an alanine). In the latter case, the antibodies are raised against the mutated version of the protein backbone, while the library is diversified using the active protein backbone as a template, and this embodiment of the method is also considered an aspect of the present invention.

In another embodiment, the antibodies can be purified using an epitope-specific ligand. In the case of linear epitopes, this can be in the form of a peptide fragment of the protein backbone, while in the case of a structural epitope this could be in the form of a peptide (or peptide phage membrane protein fusion) which has been isolated by a peptide display library using antigen-specific polyclonal antibodies, as described above. Such purification schemes lead to “monospecific antibodies” which are useful for the current invention.

In another embodiment, the antibodies can be monoclonal antibodies, each of which are considered a subgroup of “monospecific antibodies” as they have only one binding speicificity. The hybridoma clones can be selected based on specificity for the entire protein backbone or for specificity for an epitope (as described above). In either case, the epitope specificity can be assessed for instance by standard immunoassays using the isolated antibody-binding peptides in order to assign the specificity of each hybridoma clone to a particular epitope pattern. This way, one can create libraries with variation in a single epitope and assay it with one or more monoclonal antibodies specific for that particular epitope in order to get a very specific response.

Further, the antibodies, whether specific polyclonal, monospecific, or monoclonal, can be labelled to allow detection. Also, secondary antibodies directed against the primary antibodies can be labelled. The label can be a chemically bound compound such as peroxidase, streptavidin, alkaline phosphatase, fluorescent or luminescent compounds or others, or it can be a fusion tag such as a polyhistidine tag, a FLAG peptide, an S-tag or other fusion peptides for which there are or can be made specific antibodies.

When assessing antibody binding capacity of a protein variant sampled directly from a cell culture supernatant, there will be many components of the supernatant, which may interfere with the antibody-binding assay. This background interference can be reduced by thorough purification of the protein backbone prior to sensitisation of the test animal, or by several other methods. One such method is to purify the antibodies on a column with immobilized ‘cellular impurities’ obtained by culturing a strain of host cells which have not been transformed or which have been transformed with a vector that does not contain any protein backbone or protein variant, as described (Naver and Løvborg, Scand. J. Immunol., 41, pp. 443-448, 1995). Another method to reduce background interference is to raise the antibodies against a protein backbone, which has been expressed in an organism or strain different from the host cells used for expression of the diversified libraries. One could use E. coli instead of bacillus, or even different strains of Bacillus (e.g. B. subtilis vs. B. licheniformis) may be sufficiently different to ensure that polyclonal antibodies are specific for impurities from one strain, but not from the other. A third method is to use immobilization of the protein variants (as described above) to facilitate removal of cell culture supernatant impurities.

In one aspect of the invention, the antibody binding assay is multivalent in nature, i.e. it depends on multivalent or bivalent interactions between antibody and antigen. Examples of such assay formats are agglutination assays and assays based on ‘passive immunization’ of effector cells, e.g. mast cells or basophiles, with IgE antibodies and detection of cell-specific responses to antigen-induced aggregation of the cell-surface bound IgE molecules (Skov, PS et al, Pediatr. Allergy Immunol., 8, pp.-156-158, 1995; Diamant and Pratkar, Int. Archs. Allergy appl. Immun. 67, pp. 13-17, 1982). This aspect can be advantageous when several epitopes are diversified in the same library.

In the first aspect of the invention, the antibody binding capacity is determined in step (v). In order to achieve high throughput of the screening method, it is desirable to use an antibody binding analysis that requires few dosages, preferably only one dosage of protein variant, and which in other ways is designed to give the highest likelihood of identifying low immunogenic protein variants. These variations in design of the antibody binding analysis are constitutes several embodiments of the invention, as described in the following.

Immobilized Form

In one embodiment, in which the protein variants have been immobilized to a solid phase for analysis, the binding can be determined using a labelled primary antigen-specific antibody or alternatively by using a primary antigen-specific antibody and a labelled secondary species-specific anti-Ig antibody. Immobilization of the protein variant makes it easier to change the reaction medium several times, introduce washing steps etc. In one aspect of this embodiment, the antibody-binding can be determined using competitive antigen (e.g. protein backbone) and labelled antibodies in the solution.

Soluble Form

The protein variants may be analysed for antibody binding capacity directly, or they may have been immobilized to a solid phase and then eluted (as described above). In either case, the antibody binding capacity is analysed with the protein variant in solution.

In one embodiment, the antibodies have been coated onto a solid phase (such as the surface of wells in a microtiter plate. Thus, protein variants bind to (or be captured by) the immobilized antibodies at the surface, and the supernatant contain only antigens that do not bind well to the antibodies (provided that the relative amounts of coated antibody and added protein variant have been adjusted such that the protein backbone when added in similar amounts as the protein variant, binds fully to the coated antibodies). After binding has equilibrated, the supernatant is withdrawn and assayed for functionality to determine whether functional protein variants have reduced antibody-binding capacity. In this mode, the analysis of antibody binding and protein variant functionality are combined to give a single read-out. Optionally, the conditions can be adjusted to lower the affinity between antibody and protein backbone in order to allow protein variants with moderately reduced antibody binding capacity to be free in solution. The affinity can be lowered for instance by adjusting the salt concentration and/or pH or by carrying out the incubation in the presence of surface active ingredients or in the presence of competitive modified protein backbone, which has been inactivated (chemically or genetically, as described above for proteases) in order to give no signal in the functionality assay.

In another embodiment, the antibody-coated surfaces are incubated with protein variant and labelled competitive antigen in a ratio that ensures that no or minimal amounts of labelled competitor are bound to the surface when incubated with the protein backbone. After incubation, the supernatant is removed and the amount of bound competitor is determined. The advantage of this approach is that a protein variant that has had an epitope eliminated will be less likely to give a false positive signal by binding through a different epitope. If the protein variant has had an epitope eliminated the subset of antibodies that are specific for this epitope will be unoccupied and allow binding of labelled competitor to the surface, even when the labelled competitor is present in far lower concentration than the protein variant.

Determining Functionality

A wide variety of protein functionality assays are available in the literature. Especially, those suitable for automated analysis are useful for this invention. Several have been published in the literature such as protease assays (WO99/34011, Genencor International; J. E. Ness, et al, Nature Biotechn., 17, pp. 893-896, 1999), oxidoreductase assays (Cherry et al., Nature Biotechn., 17, pp. 379-384, 1999, and assays for several other enzymes (WO99/45143, Novo Nordisk).

Those assays that employ soluble substrates can be employed for direct analysis of functionality of immobilized protein variants. Especially the protease assays described in the Materials and Methods section are useful for that aspect of the invention.

Further Analysis of Selected Protein Variants

When protein variants have been selected based on the methods described in this invention, it is desirable to confirm their antibody binding capacity, functionality, and immunogenicity using a purified preparation. For that use, a selected clone should be re-isolated by conventional microbiological techniques and its characteristics tested in the same assay system, then (if results are confirmed) the protein variant of interest should be expressed in larger scale, purified by conventional techniques, and the reduced antibody binding capacity and the functionality should be examined in detail using dose-response curves and e.g. competitive ELISA (C-ELISA).

The potentially reduced allergenicity (which is likely, but not necessarily true for a variant w. low antibody binding) should be tested in in vivo or in vitro model systems: e.g. an in vitro assays for immunogenicity such as assays based on cytokine expression profiles or other proliferation or differentiation responses of epithelial and other cells incl. B-cells and T-cells. Further, animal models for testing allergenicity should be set up to test a limited number of protein variants that show desired characteristics in vitro. Useful animal models include the guinea pig intratracheal model (GPIT) (Ritz, et al. Fund. Appl. Toxicol., 21, pp. 31-37, 1993), mouse subcutaneous (mouse-SC) (WO 98/30682, Novo Nordisk), the rat intratracheal (rat-IT) (WO 96/17929, Novo Nordisk), and the mouse intranasal (MINT) (Robinson et al., Fund. Appl. Toxicol. 34, pp. 15-24, 1996) models.

The immunogenicity of the protein variant is measured in animal tests, wherein the animals are immunised with the protein variant and the immune response is measured. Specifically, it is of interest to determine the allergenicity of the protein variants by repeatedly exposing the animals to the protein variant by the intratracheal route and following the specific IgG and IgE titers. Alternatively, the mouse intranasal (MINT) test can be used to assess the allergenicity of protein variants. By the present invention the allergenicity is reduced at least 10 times as compared to the allergenicity of the parent protein, preferably 50 times reduced, more preferably 100 times.

However, the present inventors have demonstrated that the performance in a competitive ELISA correlates closely to the immunogenic responses measured in animal tests. To obtain a useful reduction of the allergenicity of a protein, the IgE binding capacity of the protein variant must be reduced to at least below 75%, preferably below 50% of the IgE binding capacity of the parent protein as measured by the performance in competitive IgE ELISA, given the value for the IgE binding capacity of the parent protein is set to 100%.

Materials and Methods

Horse Radish Peroxidase labelled anti-rabbit-Ig (Dako, DK, P217; dilution 1:1000).

Rabbit anti-Savinase polyclonal IgG prepared by conventional means.

Rat anti-Savinase IgE.

CovaLink NH2 plates (Nunc, Cat# 459439)

Cyanuric chloride (Aldrich), Acetone (Merck), Tween 20 (Merck), Skim Milk powder (Difco), H2SO4 (Merck), OPD: o-phenylene-diamine: (Kementec cat no. 4260), H202, 30% (Merck).

Buffers and Solutions:

Carbonate buffer (0.1 M, pH 10): Na2CO3 10.60 g/L.

PBS (pH 7.2): NaCl 8.00 g/L; KCl 0.20 g/L; K2HPO4 1.04 g/L; KH2PO4 0.32 g/L.

Washing buffer: PBS, 0.05% (v/v) Tween 20.

Blocking buffer: PBS, 2% (wt/v) Skim Milk powder.

Dilution buffer: PBS, 0.05% (v/v) Tween 20, 0.5% (wt/v) Skim Milk powder.

Succinyl-Alanine-Alanine-Proline-Phenylalanine-para-nitroanilide.

(Suc-AAPF-pNP) Sigma no. S-7388, Mw 624.6 g/mole.

Activation of CovaLink Plates:

A fresh stock solution of 10 mg/ml cyanuric chloride in acetone is diluted into PBS, while stirring, to a final concentration of 1 mg/ml and immediately aliquoted into CovaLink NH2 plates (100 microliter per well) and incubated for 5 minutes at room temperature. After three washes with PBS, the plates are dryed at 50° C. for 30 minutes, sealed with sealing tape, and stored in plastic bags at room temperature for up to 3 weeks.

Immobilization of Antibody/Competitive Antigen:

Activated CovaLink NH2 plates are coated overnight at 4° C. with 100 microliter of the desired protein (5 micro gram/ml) in PBS followed by 30 min incubation with blocking buffer at room temperature and four washes in PBS-tween.

Protease Activity:

Analysis with Suc-Ala-Ala-Pro-Phe-pNa:

Proteases cleave the bond between the peptide and p-nitroaniline to give a visible yellow colour absorbing at 405 nm. Briefly, 100 mg suc-AAPF-pNa is dissolved into 1 ml dimethyl sulfoxide (DMSO). 100 microliter of this is diluted into 10 ml with Britton and Robinson buffer, pH 8.3, and used as substrate for the protease. Reaction is detected kinetically in a spectrophotometer.

Analysis with BODIPY-casein:

The supernatant from the culture medium is diluted 200-fold in the reaction mixture, which contains 5 microg/ml BODIPY FL-casein (Molecular Probes), 1 mM CaCl₂, and 50 mM Tris-HCL, pH 7,5. After 60 min. incubation at room temperature, the plates are read at 520 nm with excitation at 485 nm using a FLUOstar (BMG Technologies).

### EXAMPLES

### Example 1

### Capturing Antigen

In this example a protease antigen is captured on immobilized antibodies and the non-captured fraction is assayed for functionality. This is performed on CovaLink NH2 plates coated with rat anti-Savinase IgE.

Protease libraries producing epitopee variants were established. These variants might or might not be His-tagged. Screening is directly on bacterial culture media, using covalink plates coated with mouse anti-rat IgE monoclonal antibodies satured with anti-savinase specific rat IgE. The amounts of bound wild type antigen was determined with a anti-wild type polyclonal rabbit antiserum.

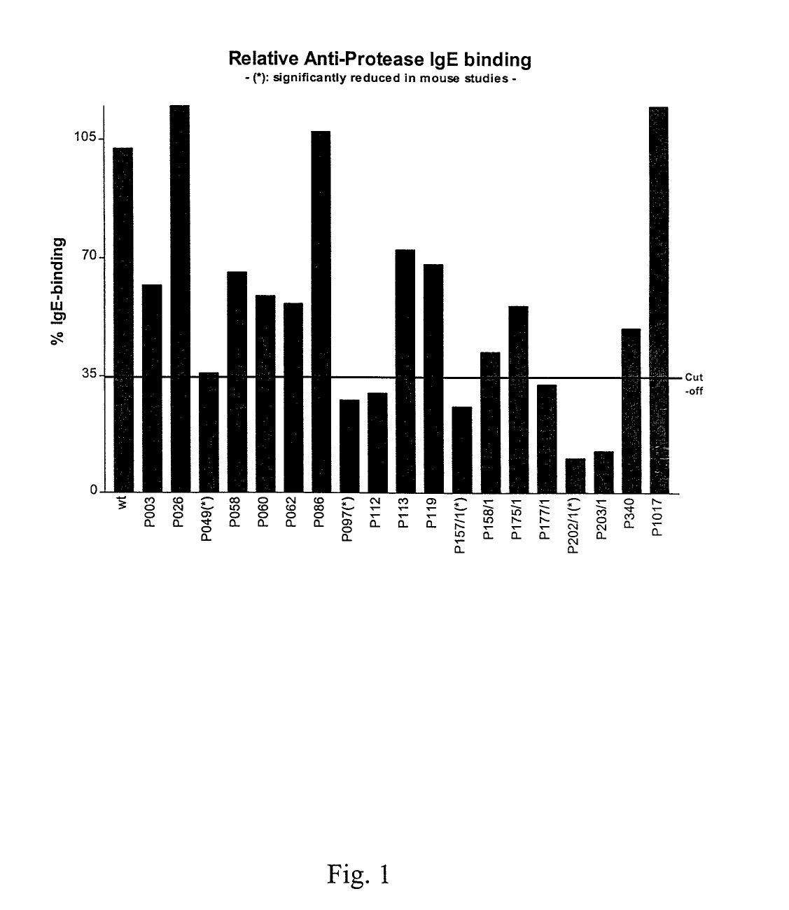

The results are shown in FIG. 1

### Example 2

### Immobilized Competitor

In this example a ‘backbone protease’ protease inhibitor is immobilized in the wells and incubated with an excess of the protein variant and labelled antibodies. The level of bound antibodies is determined.

25 microliter sample and 25 microliter anti-Savinase antibody (both diluted in PBS-tween with 0.5% (w/v) skim milk) are added to the coated well and incubated at room temperature (30 min). The supernatant is removed and the wells are washed three times in PBS-tween.

50 microliter HRP-labelled speciec-spcific anti-Ig antibody is added and incubated 30 min, then the wells are wash three times in PBS-tween. Finally, 50 microliter ODP-H202-mixture is added and A492 is measured kinetically to determine the level of bound antibodies. Dilutions are adjusted such that the ‘backbone protein’ gives none or very little level of bound antibody.

A separate sample is analysed for functionality and the two values are compared.

Desired protein variants show a high level of bound antibody and at the same time a level of functionality similar to the ‘backbone protein’.

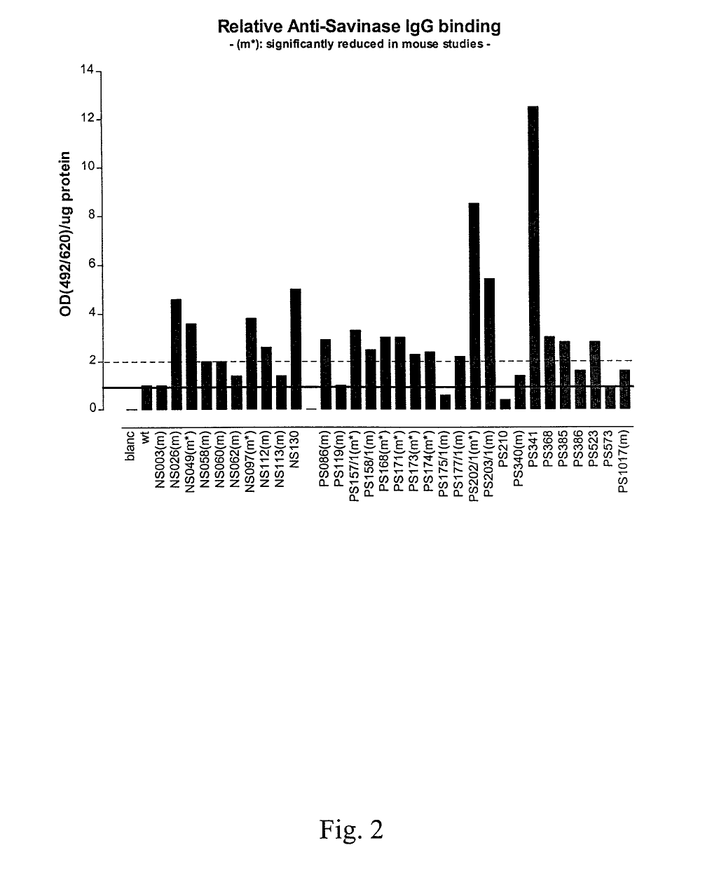

The results of the anti-savinase IgG binding is shown in FIG. 2.

### Example 3

### Immobilization of His-tagged Proteases

The DNA sequence encoding the protease Savinase® (Novo Nordisk A/S, Denmark) is translationally fused to a sequence encoding a polyhistidine tag (His6) and libraries of Savinase®-His6 variants are produced and introduced into Bacillus. After standard culturing, a limited number of Savinase® enzymes of each variant (about 10% of what is secreted by Bacillus carrying the wildtype Savinase gene) are immobilized in the wells of Ni-NTA microtiter plates. The unbound fraction including cells and excess Savinase® is removed, and the plate washed once or twice in a buffer containing 5-20 mM Imidazole.

The immobilized Savinase can now be assayed for antibody binding capacity directly, or used modified with activated PEG and washed prior to analysis. The His-tagged Savinase® variants are released from the solid support by the addition of 250 mM Imidazole, and aliquots of the supernatants from each well are sampled for antibody binding and functionality assays as described in the previous examples.

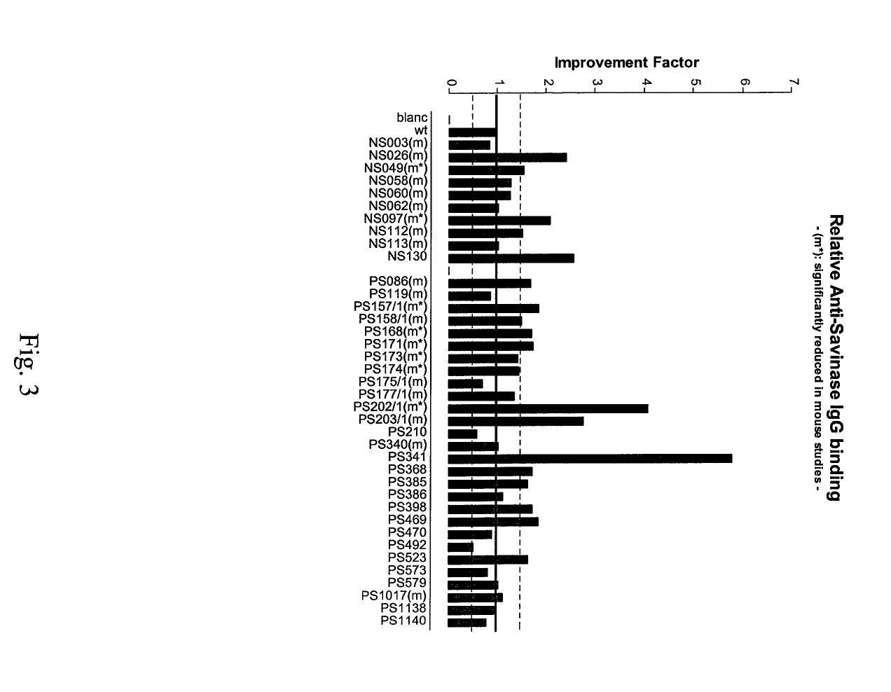

The results are shown in FIG. 3.

1. A method for screening a library of protein variants for functional variants with reduced antibody binding capacity, comprising the steps of:
(i) generating a diversified library of protein variants starting from a relevant protein backbone,
(ii) transforming the library into suitable host cells,
(iii) culturing host cells,
(iv) sampling each cell culture,
(v) analysing a sample by determining the antibody binding capacity of the variant protein,
(vi) analysing a sample by determining the functionality of the variant protein:

2. The method according to claim 1, wherein the following steps are added between step (ii) and (iii):
(iib) culturing host cells,
(iic) assaying function,
(iid) selecting host cells expressing functional protein variants.

3. The method according to claim 2, wherein the selected cells in step (iid) are picked by a colony-picker.

4. The method according to claims 1-3, wherein the library diversity is located in epitope areas.

5. The method according to claims 1-4, wherein the protein variants are modified by substitution, addition, and/or deletion of amino acid residues suitable for chemical modification of the protein.

6. The method according to claims 1-2, wherein the protein variants are modified by introduction of one or more additional post-translational modification site and expressed in a host suitable for the corresponding in vivo post-translational modification.

7. The method according to claim 6, wherein the site is a N-glycosylation site or a phosphorylation site.

8. The method according to claims 1-7, where the diversified library is randomized at one or more individual positions (DNA codons) at the primer level.

9. The method according to claim 8, wherein the library is biased towards amino acids that can be chemically modified.

10. The method according to claim 8, wherein the library is biased towards amino acids that correspond to post-translational modification recognition sequences.

11. The method according to claim 10, wherein the amino acids correspond to N-glycosylation sites or phosphorylation sites.

12. The method according to claim 8, wherein the library is biased towards sequences that are not predicted to result in formation of new epitopes.

13. The method according to claims 1-7, where the diversified library is randomized by combination of segments of known sequence,

14. The method according to claims 1-13, wherein the library is diversified simultaneously at several discrete sites on the three dimensional structure.

15. The method according to claim 14, wherein the library is assayed with specific polyclonal antibodies.

16. The method according to claim 14. Wherein the library is assayed for antigen binding in an assay that requires bivalent antigen-antibody interactions.

17. The method according to claims 1-13, wherein the library is diversified at a single site on the three-dimensional structure.

18. The method according to claim 17, wherein the library is assayed with a monospecific antibody.

19. The method according to claim 17, wherein the library is assayed with a monoclonal antibody.

20. The method according to claims 19, wherein the library is diversified at a single epitope area and assayed with a monospecific antibody purified using the corresponding peptide-phage membrane protein fusion.

21. The method according to claims 1-20, wherein the cells in step (ii) are dispensed in a multi-compartment device in a dilution such that each compartment contains an average of 0,2-1,0 cells.

22. The method according to claims 1-21, wherein the sample of step (iv) is separated from the host cells by a membrane process.

23. The method according to claims 1-22, wherein the sample is analysed by determining the total content of protein variant.

24. The method according to claims 1-22, wherein the sample is analysed by exposure to adverse conditions prior to determining the functionality.

25. The method according to claims 1-22, wherein the sample functionality is analysed both prior to and after exposure to adverse conditions.

26. The method according to claims 1-13 and 21-25, wherein the antibodies are derived from animals sensitized with the backbone protein of step (i).

27. The method according to claims 26, wherein the antibodies are derived from animals sensitized by intratracheal exposure

28. The method according to claims 1-13 and 21-25, wherein the antibodies are derived from human volunteers that are sensitized to the backbone protein of step (i).

29. The method according to claims 26-28, wherein the antibodies are raised against the same protein, but expressed in a strain different from the host cells, such as to minimize background binding to host cell impurities.

30. The method according to claims 26-29, wherein the antibodies are contained in serum from the animal or human.

31. The method according to claim 30, wherein the antibodies are IgG, IgM and/or IgE antibodies.

32. The method according to claims 30-31, wherein the antibodies are antigen-specific antibodies.

33. The method according to claim 32, wherein the antibodies are selected for the binding affinity to specific epitopes.

34. The method according to claims 30-33, wherein the antibodies are purified by capturing those that bind to impurities of the culture supernatant.

35. The method according to claims 26-29, wherein the antibodies are monoclonal antibodies.

36. The method according to claim 35, wherein the clones are selected for the binding affinity of their corresponding antibodies to specific epitopes.

37. The method according to claims 1-36, wherein the antibody binding is determined from a single dilution of the protein variant.

38. The method according to claims 1-37, wherein the functionality to be determined is enzyme activity.

39. The method according to claims 1-38, wherein the protein variants are bound to a solid phase.

40. The method according to claim 39, wherein the solid phase is a dipstick.

41. The method according to claim 40, wherein the immobilised protein variants are transferred from one test solution to another by sequentially immersing the dipstick in the test solutions.

42. The method according to claim 41, wherein the test solution(s) is (are) placed in wells, e.g. in 96 well plates.

43. The method according to claim 39, wherein the solid surface is a microtiter well surface.

44. The method according to claim 39, wherein the solid surface is the surface of beads.

45. The method according to claims 39-44, wherein the binding capacity of the solid surface is less than the average protein variant content of the sample such that the surface binding samples a reproducible amount of protein variant for analysis.

46. The method according to claims 39-45, wherein the protein variant is linked to a fusion peptide which mediates binding to the solid phase.

47. The method according to claims 39-46, wherein the protein variant is modified by chemical conjugation prior to steps (v) and (vi).

48. The method according to claim 47, wherein the protein variant is conjugated with activated PEG.

49. The method according to claim 48, wherein the protein is conjugated to activated PEG molecules of molecular weight ranging from 100 to 5000 Da at a ratio of activated polymer to lysines in protein that is greater than 5.

50. The method according to claims 39-49, wherein the test solution of step (v) comprises antibodies and competitive backbone protein.

51. The method according to claims 39-50, wherein bound antigen is detected using a primary antigen-specific antibody and a labelled secondary antibody specific for the primary antibody.

52. The method according to claims 39-50, wherein the bound antigen is detected using a labelled primary antibody.

53. The method according to claim 50, wherein the competitor is added in an amounts equal to the amount of immobilised protein variant, better at amounts that are 10× higher than the amount of immobilised protein variant, but preferentially more than 100× higher than the amount of immobilised protein variant.

54. The method according to claim 50 or 53, wherein the selected variants show reduced antibody binding in the presence of competitor, at least 5% reduced, preferably more than 10% reduced, more preferred above 50% reduced and most preferably more than 75% reduced.

55. The method according to claims 39-54, wherein the protein variant is eluted from the solid phase prior to determining the functionality in step (vi).

56. The method according to claims 1-38, wherein the antibody binding is determined by agglutination of beads or cells coated with antibodies.

57. The method according to claims 1-38, wherein the antibody binding is determined by IgE antibodies bound to the surface of effector cells

58. The method according to claim 39-54, wherein the protein variants are bound reversibly to a solid phase and are eluted prior to analysis in (v) and (vi).

59. The method according to claims 1-38 or claim 58, wherein antibodies are coated on a solid surface and incubated with sample.

60. The method according to claim 59, wherein labelled competitive protein is incubated together with sample.

61. The method according to claim 60, wherein the amount of competitor is smaller than the average amount of protein variant in the sample.

62. The method according to claims 60-61, wherein the competitor is labelled chemically or genetically.

63. The method according to claims 60-62, wherein bound labelled competitor is determined after removal of the sample.

64. The method according to claims 1-38 or claim 58, wherein antibody binding is used to capture protein variant and the functionality is determined for the non-captured fraction.

65. The method according to claim 64, wherein binding is performed at conditions where antigen-antibody affinity is lowered, such that a moderate change in affinity may lead to non-capture.

66. The method according to claims 64-65, wherein binding is performed in the presence of inactivated competitor, such that a small change in affinity may lead to non-capture.

67. The method according to claim 66, wherein competitor is inactivated chemically, such that it will not affect the functionality assay.

68. The method according to claim 66, wherein the competitor is inactivated by protein engineering, such that it will not affect the functionality assay.
